(19) United States
(12) Patent Application Publication
(10) Pub. No.: US 2024/0366929 A1
(43) Pub. Date: Nov. 7, 2024

(54) DETECTING OPERATIONAL CONDITIONS OF MECHANICAL CIRCULATORY SUPPORT DEVICES

(71) Applicant: Mayo Foundation for Medical Education and Research, Rochester, MN (US)

(72) Inventors: Barry A. Boilson, Rochester, MN (US); Dan Dragomir-Daescu, Rochester, MN (US); Alfredo L. Clavell, Rochester, MN (US); John M. Stulak, Rochester, MN (US); Kent D. Carlson, Rochester, MN (US)

(21) Appl. No.: 18/683,400

(22) PCT Filed: Aug. 24, 2022
(86) PCT No.: PCT/US2022/041361

§ 371 Date: Feb. 13, 2024

Related U.S. Application Data
(60) Provisional application No. 63/236,469, filed on Aug. 24, 2021.

(51) Int. Cl. A61M 60/546; A61M 60/178; A61M 60/232; A61M 60/403

(52) U.S. Cl. CPC A61M 60/546; A61M 60/178; A61M 60/232; A61M 60/403; A61M 2205/3375

(57) ABSTRACT

Described herein are systems and methods for assessing a condition of a mechanical circulatory support device (MCSD). The systems and methods can be used for early detection of device malfunction. The system can include a sensing subsystem that can obtain from one or more sensors signals indicative of at least one of vibrations or acoustics of the MCSD in operation in the mammal. The system can also include a computing subsystem. The computing subsystem can receive the signals indicative of at least one of the vibrations or the acoustics of the MCSD in operation in the mammal. process the signals to identify at least one harmonic in the signals, and evaluate the condition of the MCSD using the at least one harmonic identified in the signals indicative of at least one of the vibrations or the acoustics of the MCSD in operation in the mammal.

### CROSS-REFERENCE TO RELATED APPLICATIONS

This application claims the benefit of U.S. Provisional Application Ser. No. 63/236,469, filed Aug. 24, 2021. The disclosure of the prior application is considered part of (and is incorporated by reference in) the disclosure of this application.

### TECHNICAL FIELD

This document describes technology related to mechanical circulatory support devices (MCSDs), and particularly techniques for detecting operational conditions of MCSDs.

### BACKGROUND

Mechanical circulatory support devices (MCSDs), such as Left Ventricular Assist Devices (LVADs), have revolutionized the care of patients with end-stage heart failure. LVADs can be considered a long-term alternative to cardiac transplantation for ineligible patients, and can be an effective and durable bridge to transplantation on those who are eligible. However, LVAD pump thrombosis can be a significant and potentially devastating complication that can sometimes lead to stroke, pump stoppage, thromboembolic complications, and even death. Clinical presentation of pump thrombosis is often late with complications already evident, at which point the only effective therapy is pump exchange with significant perioperative morbidity and mortality.

### SUMMARY

The document describes techniques for assessing operational conditions of MCSDs. The techniques can be applied to realize early detection of pump thrombosis and LVAD malfunctions, e.g., using machine learning models. Early detection of pump thrombosis can enable earlier intervention with augmented anticoagulation and antiplatelet therapy to avoid serious complications and need for pump exchange.

Analysis of very high frequency harmonic signals in LVAD recipients can demonstrate significant differences with the onset of thrombosis and other forms of device malfunction. The relative amplitude of higher order harmonics can increase in the presence of pump thrombosis. A triaxial accelerometer or surface hydrophone and signal acquisition software with a very high sampling rate can detect higher frequency harmonic signals consistently and reproducibly. Accordingly, the disclosed technology can provide for using a triaxial accelerometer to obtain high resolution high frequency recordings with a low signal to noise ratio and at higher sampling rates from LVAD recipients with and without suspected pump thrombosis. The disclosed technology can also use machine learning based analysis techniques specifically adapted to non-linear high frequency harmonic signals to detect differences present in the high frequency recording to identify pump thrombosis. Machine learning models can, for example, be trained to identify high frequency harmonics that are made by the LVAD or other MCSD while in operation that are indicative of pump thrombosis or other device malfunctions (e.g., abnormal operational conditions).

The disclosed technology, including triaxial accelerometer hardware, can be incorporated into LVAD or other MCSD designs. The disclosed technology can also be integrated with software for signal analysis and interpretation that can further be used by LVAD or other MCSD microprocessors to generate warning signals and alarms about operational conditions of such devices. The disclosed technology can also apply to handheld detection devices that use external surface scanning to determine whether the LVAD or other MCSD is operating in abnormal conditions. Although the disclosure is described in reference to LVADs, the disclosed techniques are also applicable to other types of MCSDs.

One or more embodiments described herein can include a system for assessing a condition of a mechanical circulatory support device (MCSD) in a mammal, the system including a sensing subsystem and a computing subsystem. The sensing subsystem can obtain from one or more sensors signals indicative of at least one of vibrations or acoustics of the MCSD in operation in the mammal. The computing subsystem can receive the signals indicative of at least one of the vibrations or the acoustics of the MCSD in operation in the mammal, process the signals to identify at least one harmonic in the signals, and evaluate the condition of the MCSD using the at least one harmonic identified in the signals indicative of at least one of the vibrations or the acoustics of the MCSD in operation in the mammal.

In some implementations, the embodiments described herein can further include one or more of the following features.

For example, the at least one harmonic can be at least one of a fundamental frequency (FF), second harmonic (2H), third harmonic (3H), fourth harmonic (4H), and sixth harmonic (6H). The one or more sensors can include at least one of a microphone, a triaxial accelerometer, and a hydrophone. Processing the signals can include filtering out extraneous noise from the signals. As another example, evaluating the condition of the MCSD can include determining that an absolute amplitude of the at least one harmonic identified exceeds a threshold level. The at least one harmonic can be at least one of a 2H and a 3H.

As another example, evaluating the condition of the MCSD can include determining that a relative amplitude of the signals exceeds a threshold level. The relative amplitude can be a ratio of at least one harmonic to an amplitude of a FF in the signals. The relative amplitude can also be the ratio of the amplitude of at least one harmonic to the average power of the time-domain signal.

In some implementations, the one or more sensors can be positioned on the mammal's chest at a left lower sternal border. In some implementations, the one or more sensors can be attached to a handheld surface detection device. In yet some implementations, the one or more sensors can be attached to the MCSD. The one or more sensors can include at least one of an electronic stethoscope, a hydrophone, an accelerometer, an ECG sensor, and an echocardiographic sensor.

As another example, processing the signals to identify at least one harmonic in the signals can include aurally identifying FF, 2H, and 3H as audible tones, reproducing the audible tones into generated tones using a signal generator, superimposing the generated tones into a sound recording, fine tuning frequency of the generated tones in the sound recording by identifying beats, identifying visual waveforms based on playing back the sound recording, performing fast fourier transformation (FFT) on the sound recording, labeling frequencies in the sound recording, and labeling higher order harmonics that are inaudible but are identified based on performing the FFT.

In some implementations, the computing subsystem can also determine, based on evaluating the condition of the MCSD, that the MCSD has pump thrombosis, and output, at a user device, an alert notification identifying the pump thrombosis of the MCSD. As another example, the MCSD can be a left ventricular assist device (LVAD). As yet another example, the sensing subsystem can also obtain from the one or more sensors signals indicative of electromagnetic signals of the MCSD in operation in the mammal.

As another example, the computing subsystem can evaluate the condition of the MCSD using the at least one harmonic identified based on retrieving, from a data store, one or more machine learning models, and classifying, based on applying the one or more machine learning models, the at least one harmonic as corresponding to normal or abnormal operational conditions of the MCSD. The one or more machine learning models could have been trained using training datasets that may include signals labeled with normal operational conditions and signals labeled with abnormal operational conditions. In some implementations, the abnormal operational conditions of the MCSD can include at least one of presence of pump thrombosis and likelihood to develop pump thrombosis at the MCSD.

One or more embodiments described herein can also include a method for assessing a condition of a mechanical circulatory support device (MCSD) in a mammal, the method including receiving, from one or more sensors, signals indicative of at least one of vibrations or acoustics of the MCSD in operation in the mammal, processing the signals to identify at least one harmonic in the signals, and evaluating the condition of the MCSD using the at least one harmonic identified in the signals indicative of at least one of the vibrations or the acoustics of the MCSD in operation in the mammal.

The method described herein can optionally include one or more of the abovementioned features. The method can also include one or more additional features. For example, evaluating the condition of the MCSD using the at least one harmonic identified can include retrieving, from a data store, one or more machine learning models, and classifying, based on applying the one or more machine learning models, the at least one harmonic as corresponding to normal or abnormal operational conditions of the MCSD. The one or more machine learning models could have been trained using training datasets that may include signals labeled with normal operational conditions and signals labeled with abnormal operational conditions.

The devices, system, and techniques described herein may provide one or more of the following advantages. For example, the disclosed techniques can be used for early detection of pump thrombosis or other malfunctions in LVAD and other MCSD technology. Early detection can help avoid stroke, pump stoppage, and death that may otherwise occur when pump thrombosis or other malfunctions are not identified early on. Therefore, the disclosed technology can be beneficial to protect a recipient's health, safety, and life.

As another example, using machine learning and triaxial accelerometers, the disclosed techniques can provide for consistent and accurate detection of acoustic characteristics indicative of abnormal operational conditions. Current technologies may not detect pump thrombosis or other malfunctions since such technologies may not be able to consistently detect higher order frequencies. Here, models can be trained using machine learning algorithms and labeled training data sets to detect varying higher order frequencies indicative of different types of malfunctions and operational conditions. The disclosed techniques can therefore provide for more accurate and consistent classification of LVAD or other MCSD operational conditions. Such classifications can be provided to a user such that the user can take appropriate action to rectify the operational conditions of the LVAD or other MCSD before any harm is done to the recipient.

As another example, machine learning algorithms can provide for continuing analysis of subtle, low amplitude parameters over time. After all, higher order harmonics, beyond those which can be detected by the human ear, can likely be altered in relative amplitude in the setting of pump thrombosis or device malfunction. The disclosed technology therefore provides continuous monitoring and comparing of subtle, low amplitude parameters over time to baseline conditions in order to detect pump thrombosis and/or mechanical malfunction early, before overt manifestations become evident.

The details of one or more implementations are set forth in the accompanying drawings and the description below. Other features and advantages will be apparent from the description and drawings, and from the claims.

### BRIEF DESCRIPTION OF THE DRAWINGS

FIG. 1A is a conceptual diagram of a system for evaluating an operational condition of a MCSD.

FIG. 1B is a system diagram depicting computer components that can be used to perform the techniques described herein.

FIGS. 2A-B is a flowchart of a process for evaluating an operational condition of a MCSD.

FIG. 3 illustrates a process for identifying frequencies from an LVAD sound recording.

FIG. 4 depicts a table demonstrating differences among patients with and without thrombosis with different exemplary LVAD types.

FIG. 5 depicts a table describing exemplary findings at pump disassembly where thrombosis was suspected.

FIG. 6A-C depicts graphical representations of Fast Fourier transformations on sound recordings for three example LVADs.

FIGS. 7A-C depict graphical representations of Spearman rank correlations between pump seeds and frequencies for each pump type in three example LVADs.

FIG. 8A depicts graphical representations of amplitudes of frequencies by pump type and suspicion for thrombosis.

FIG. 8B depicts graphical representations comparing absolute amplitudes of frequencies where thrombosis is present or absent.

FIG. 8C depicts graphical representations comparing relative amplitudes of frequencies where thrombosis is present or absent.

FIG. 9A illustrates visual analysis of waveforms in example LVADs where thrombosis is present or absent.

FIG. 9B illustrates Fast Fourier transformations of sine and sawtooth waves.

FIG. 10 is a high frequency color map comparing an electronic stethoscope (labeled “microphone”), Z-axis component of a triaxial accelerometer, uniaxial accelerometer, and hydrophone.

FIG. 11 is a low frequency color map comparing an electronic stethoscope (labeled ‘microphone’), Z-axis component of a triaxial accelerometer, uniaxial accelerometer, and hydrophone.

FIG. 12 depicts time frequency analysis of signals acquired from the Z-axis component of a triaxial accelerometer.

FIG. 13 depicts frequency envelope spectrum analysis of repeating signals acquired from the Z-axis component of a triaxial accelerometer.

FIG. 14 depicts time frequency analysis of signals acquired from a hydrophone.

FIG. 15 depicts frequency envelope spectrum analysis of repeating signals acquired from a hydrophone.

FIG. 16 is a 2D color doppler imaging in an apical 2 chamber view that demonstrates LVAD derived interference.

FIG. 17 is a pulsed wave doppler imaging in an apical 2 chamber view that demonstrates waveform signals from the doppler interference identified in FIG. 16.

FIG. 18 depicts ECG demonstrating LVAD derived electrical noise.

FIG. 19 is a schematic diagram that shows an example of a computing device and a mobile computing device.

### Like reference symbols in the various drawings indicate like elements.

### DETAILED DESCRIPTION

This document describes systems, methods, devices, and techniques for evaluating conditions of MCSDs, such as LVADs. This disclosure provides for analyzing acoustic, vibration, and/or electromagnetic signals that are generated by the MCSD when operating inside, on, or for a recipient (e.g., a mammal, such as a human patient or an animal). These signals can be detected by one or more sensors and communicated to a computing system for processing. The computing system can process the signals to perform tasks such as classifying the signals as indicating normal or abnormal operational conditions for the MCSD. In some implementations, the signals are processed using machine-learning models that have been trained to perform classification or other prediction tasks. The models can also be used to determine what type of abnormal operational conditions are detected from the signals, such as pump thrombosis, onset of pump thrombosis, or other conditions that negatively impact operation of the MCSD. The computing system can also determine and generate output indicating a condition of the respective MCSD. A user, such as a clinician or other relevant stakeholder, can use the output to determine one or more changes, modifications, or other actions that should be taken in order to ensure that the MCSD operates properly and does not negatively impact health, safety, and/or life of the MCSD recipient. The disclosed techniques are described in reference to LVADs. However, the disclosed techniques are also applicable to any other type of MCSDs.

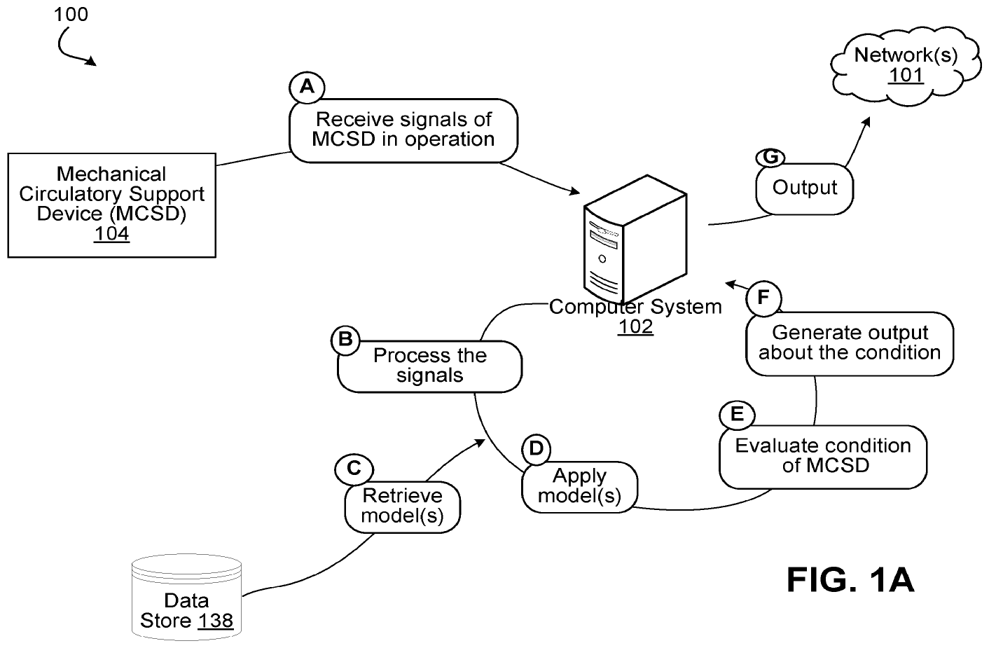

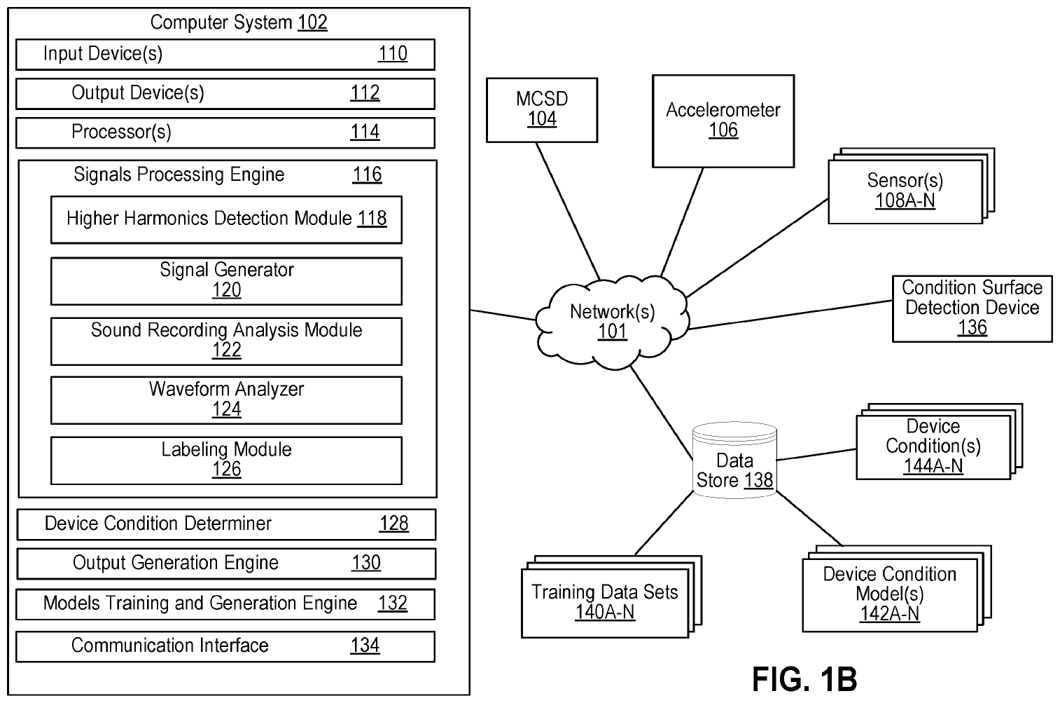

Referring to the figures, FIG. 1A is a conceptual diagram of a system 100 for evaluating an operational condition of a MCSD 104. Evaluating the operational condition of the MCSD 104 can include assessing a condition that may encompass, but is not limited to, detecting pump thrombosis, device malfunctions, normal conditions, abnormal conditions, etc. A computer system 102, the MCSD 104, and a data store 138 can communicate (e.g., wired and/or wireless) via network(s) 101. The computer system 102 can be a remote computer, server, device, and/or network of servers or computers. For example, the computer system 102 can be a cloud-based service. In general, computer system 102 can include one or more computers in one or more locations. The MCSD 104 can be any mechanical circulatory support device that can be implanted or otherwise used by humans and/or animals (e.g., mammals). As an example throughout this disclosure, the MCSD 104 can be an LVAD.

The computer system 102 can receive signals of the MCSD 104 in operation (step A). For example, one or more sensors can be used to detect acoustic, vibration, and/or electromagnetic signals that are generated by the MCSD 104 when in operation/use. The sensors can be attached to or otherwise integrated into the MCSD 104. The sensors can additionally or alternatively be attached to a separate device capable of detecting signals emitted by the MSCD 104, e.g., acoustic and/or electromagnetic signals detected when a handheld device is located near the MCSD 104. The sensors additionally or alternatively be attached to or worn by a recipient of the MCSD 104.

The computer system 102 can then process the signals (step B). Processing the signals can include filtering out extraneous tones or noise from the signals and/or identifying different frequencies and harmonics in the signals. For example, the computer system can identify a fundamental frequency (FF), second harmonic (2H), third harmonic (3H), fourth harmonic (4H), sixth harmonic (6H), and higher order harmonics. Thus, the computer system 102 can identify higher order frequencies that otherwise may be challenging to identify in the signals. The computer system 102 can also determine and/or generate waveforms for the signals and/or identified harmonics. These waveforms can be analyzed by the computer system 102 to evaluate the operational condition of the MCSD 104.

Next, the computer system 102 can access one or more models from the data store 138 (step C). The models can be trained using machine learning algorithms and techniques. The models can be trained by the computer system 102 or another system, such as a remote training computer system. The models can be one or more of neural networks, convolutional neural networks (CNN), random forests, support vector machines (SVMs), etc. The models can be trained to classify signals (e.g., the signals received in step A) as corresponding to normal or abnormal operational conditions of the MCSD 104 and other types of MCSDs. Refer to FIG. 1B for further discussion about training the models.

The computer system 102 applies the model(s) in step D. Thus, the computer system 102 can provide the processed signals, or output from processing the signals, as input into the model(s). As described further below, the model(s) can be trained to analyze the processed signals or output from processing the signals to determine a condition of the MCSD 104. The inputs to the model(s) can also be digitized versions of the raw acoustic-vibration signals that are received in step A. The inputs may also be pre-processed to reflect only certain harmonics that the model(s) is trained to identify and analyze.

Accordingly, the computer system 102 can evaluate the condition of the MCSD 104 (step E). Evaluating the condition can include assessing output from applying the model(s) in step D. For example, output from a model can include a confidence score indicating a likelihood that the MCSD 104 is not operating normally. The computer system 102 can determine whether the confidence score exceeds some threshold value. If the confidence score does, the computer system 102 can determine that the MCSD 104 is likely operating in an abnormal condition. Therefore, the computer system 102 can evaluate the condition of the MCSD 104 by determining whether the MCSD 104 is operating in normal conditions or abnormal conditions and/or whether the MCSD 104 is trending towards or likely to operate in abnormal conditions. Thus, the model(s) can generate other types of output that can indicate presence or no presence of pump thrombosis and likelihood that the MCSD 104 will develop pump thrombosis. The computer system 102 can use such output to evaluate the condition of the MCSD 104.

The computer system 102 can also use output from the model(s) and evaluation of the MCSD's condition (steps D and E) to determine what type of notification, message, alert, or other output to provide to a relevant user or stakeholder (step F). For example, if the computer system 102 determines that pump thrombosis is likely to develop in the MCSD 104 (step E), then the computer system 102 can generate output such as a notification that lets the clinician know that they should service the pump sometime soon (step F). As another example, if the computer system 102 determines that pump thrombosis or other mechanical malfunction is present, then the computer system 102 can generate output such as an alert requiring immediate attention and pump replacement by the relevant user. As yet another example, if the computer system 102 determines that the MCSD 104 is operating normally, the computer system 102 may not generate any output to be presented to the relevant user. Instead, the computer system 102 can merely store an indication in the data store 138 that identifies the MCSD 104 as operating normally at the time of evaluation. In some implementations, the computer system 102 can store an indication of the condition of the MCSD 104 in the data store 138, whether the indication identifies the MCSD 104 as operating normally or abnormally. The computer system 102 can also flag an identified problem or condition or a potential problem or condition in the MCSD 104. The flagged problem or condition can then be reassessed/evaluated at a later time (e.g. automatically by the computer system 102 and/or by a relevant user, such as a clinician)

Finally, the computer system 102 can provide the generated output in step G. The computer system 102 can, for example, transmit the generated output to a user device in step G. The output can then be presented in a graphical user interface (GUI) display at the user device. Sometimes, the output can be presented at the computer system 102. Other times, the output can be presented at the user device, which is separate and/or remote from the computer system 102. In yet some implementations, outputting the generated output in step G can include storing the output in the data store 138 or another data store, database, and/or cloud-based service. Outputting in step G can also include transmitting the generated output to another computer system for additional processing and/or analysis.

FIG. 1B is a system diagram depicting computer components that can be used to perform the techniques described herein. The computer system 102 can communicate with one or more other devices via the network(s) 101. For example, the computer system 102 can communicate with the MCSD 104, an accelerometer 106, sensors 108A-N, a condition surface detection device 136, and the data store 138.

The MCSD 104 can be any mechanical circulatory support device that can be used by and/or implanted in a mammal, such as a human and/or an animal. The MCSD 104 can be any of the LVADs described throughout this disclosure.

The accelerometer 106 can be a triaxial accelerometer. The accelerometer 106 can be configured to detect acoustic, vibration, and/or electromagnetic signals that are produced by the MCSD 104 when the MCSD 104 is in operation. As described further below, the triaxial accelerometer can detect higher order frequencies that are indicative of pump thrombosis or other malfunctions (e.g., abnormal operational conditions) of the MCSD 104.

The computer system 102 can be in wired and/or wireless communication with the accelerometer 106. The accelerometer 106 can be attached to or otherwise integrated in the MCSD 104. The accelerometer 106 can also be attached to or placed on a portion of the MCSD 104 recipient's body near an implant location of the MCSD 104. Sometimes, the accelerometer 106 can be part of another device, such as the condition surface detection device 136, that can be configured to detect signals that are produced by the MCSD 104 in operation.

In some implementations, the computer system 102 may not use the accelerometer 106. In some implementations, the computer system 102 may use the accelerometer 106 but none of the sensors 108A-N. In yet some implementations, the computer system 102 can use a combination of the accelerometer and one or more of the sensors 108A-N.

The sensors 108A-N can include any variety of sensors configured to detect signals that are produced by the MCSD 104 while the MCSD 104 is operating. The sensors 108A-N can be, like the accelerometer 106, attached to the MCSD 104, worn by the MCSD 104 recipient (e.g., positioned on the recipient's chest at a left lower sternal border), and/or part of the condition surface detection device 136. The sensors 108A-N can include, but are not limited to, an electronic stethoscope, a hydrophone, an accelerometer, an electrocardiogram (ECG) sensor, and an echocardiographic sensor. As described further below, the hydrophone can be beneficial to consistently detect higher order harmonic frequencies that are produced by the MCSD 104 during operation. Therefore, the hydrophone can be used to more accurately and consistently detect changes in the signals that are indicative of abnormal operational conditions of the MCSD 104. The hydrophone can be part of the condition surface detection device 136.

The condition surface detection device 136 can be configured to detect and record signals that are produced by the MCSD 104 when in operation. The device 136 can be beneficial because it can be used with existing MCSDs for quick, easy, and accurate detection of signals that are produced by the MCSDs in operation. A user can, for example, wave or place the device 136 over a portion of the MCSD 104 recipient's body where the MCSD 104 is implanted. The device 136 offers non-invasive option to detect the signals produced by the MCSD 104 that can be analyzed by the computer system 102.

As described in reference to FIG. 1A, the data store 138 can store information about used and/or generated by the computer system 102. The data store 138 can be part of the computer system 102. The data store 138 can also be separate from any of the components depicted and described in FIG. 1B. The data store 138 can be a cloud-based storage service or other remote data store, database, and/or data repository.

The data store 138 can house training data sets 140A-N, device condition model(s) 142A-N, and device condition(s) 144A-N. The training data sets 140A-N can comprise large data sets of training samples. The training data sets 140A-N can indicate, for each training sample, whether a signal of the sample corresponds to a normal condition or an abnormal condition of a training MCSD. For example, some signals in the training data sets 140A-N can be taken from LVADs where pump thrombosis was detected by other options (e.g., by testing the LVAD, while the LVAD was used in real-time, etc.). Some of the training signals can be taken from LVADs that operated properly (no pump thrombosis or other abnormal operational conditions). Some of the training signals can be annotated and labeled by the computer system 102 or another computer system. Some of the training signals can be annotated and labeled by a human, such as a clinician or other professional.

The device condition model(s) 142A-N can be generated by the computer system 102 or another computer system. The models 142A-N can be generated using machine learning algorithms and techniques and based on the training data sets 140A-N. The models 142A-N can be any type of machine learning model, including but not limited to CNNs, random forests, and SVMs.

The device condition(s) 144A-N can include signals that are produced by the MCSD 104 and detected by the accelerometer 106, sensors 108A-N, and/or device 136. The device conditions 144A-N can therefore include raw, unprocessed acoustic, vibration, and/or electromagnetic signals that are produced by the MCSD 104 during operation. The device conditions 144A-N can also include signals and/or harmonics that are identified and/or generated by the computer system 102 using the raw, detected signals. The device condition(s) 144A-N can further include any evaluations, determinations, and/or output that are made/generated by the computer system 102 based on analyzing the signals that are produced by the MCSD 104 during operation.

The computer system 102 can include a variety of components for performing the techniques described herein. For example, the computer system 102 can include input device(s) 110, output device(s) 112, processor(s) 114, signals processing engine 116, device condition determiner 128, output generation engine 130, models training and generation engine 132, and communication interface 134.

The input device(s) 110 can include one or more devices configured to receive input signals or other information. The input device(s) 110 can include but is not limited to keyboards, touch screens, other displays, microphones, and mice. The input device(s) 110 can receive user input. For example, a clinician can provide input to the computer system 102 that indicates selection of signals produced by the MCSD 104 to be processed and analyzed. The clinician can also provide input to the computer system 102 that indicates actions to take based on the computer system 102's evaluation of the MCSD 104's condition.

The output device(s) 112 can include one or more devices for outputting or presenting information. The output device(s) 112 can include but is not limited to display screens, touch screens, pagers, speakers, and other devices. The output device(s) 112 can display information to relevant users, such as the computer system 102's evaluation of the condition of the MCSD 104.

The processor(s) 114 can be configured to execute any of the operations described herein.

The signals processing engine 116 can be configured to receive signals that are detected by the accelerometer 106, sensors 108A-N and/or condition surface detection device 136 and process such signals. In some implementations, the signals processing engine 116 may also retrieve previously captured signals from the data store 138 for processing and analysis by the computer system 102. The signals processing engine 116 can include a higher harmonics detection module 118, a signal generator 120, a sound recording analysis module 122, a waveform analyzer 124, and a labeling module 126.

The higher harmonics detection module 118 can be configured to identify, in the signals, one or more of FF, 2H, 3H, 4H, and 6H. The signal generator 120 can reproduce audible tones, such as the FF, 2H, and 3H. The sound recording analysis module 122 can superimpose the reproduced tones with the signals in order to fine tune frequencies of the reproduced tones and identify the corresponding beats. The FF, 2H, and 3H frequencies can therefore be identified and validated/confirmed. The waveform analyzer 124 can be configured to play back the signals with the reproduced tones and generate visual waveforms based on playback. The visual waveforms can be analyzed by the computer system 102 to evaluate the condition of the MCSD 104. The labeling module 126 can perform Fast Fourier transformation (FFT) on the visual waveforms such that frequencies can be identified and labelled. Sub and higher order harmonics, such as the 4H, 6H and higher, can be labelled. Although such harmonics may be inaudible, they are identified and seen on the FFT.

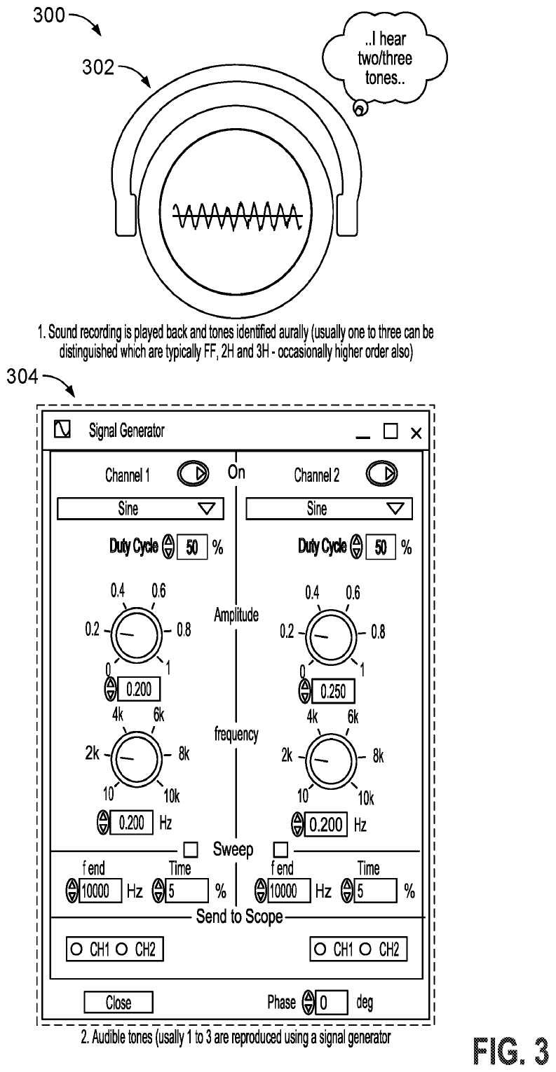

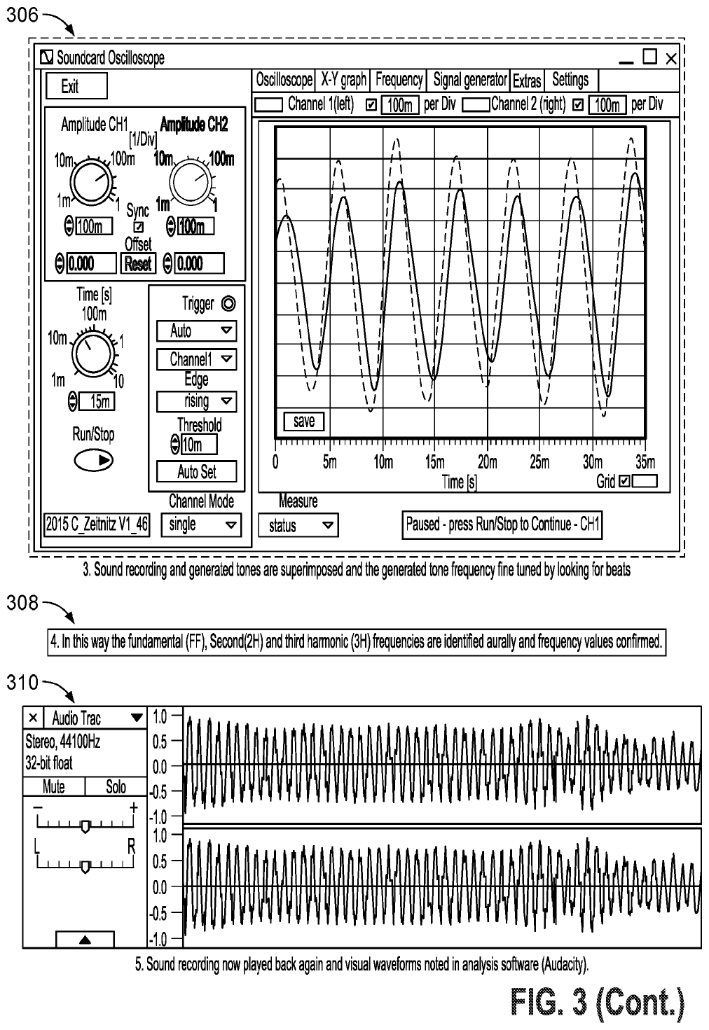

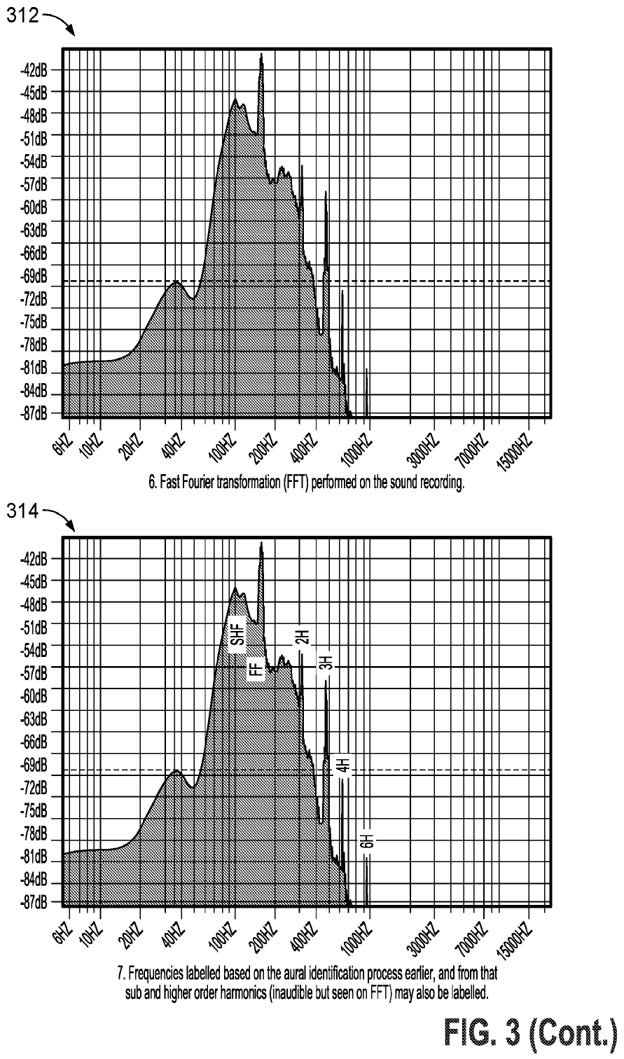

The signals processing engine 116 can include additional or fewer components to perform the techniques described herein. Refer to FIG. 3 for further discussion on how the components 116, 118, 120, 122, 124, and 126 operate to process the signals produced by the MCSD 104.

The device condition determiner 128 can be configured to evaluate the condition of the MCSD 104. The determiner 128 can use the raw signals detected by the accelerometer 106, the sensors 108A-N, and/or the device 136 to evaluate the MCSD 104's condition. The determiner 128 can also use the processed signals from the signals processing engine 116 to evaluate the MCSD 104's condition. The determiner 128 can retrieve one or more of the device condition models 142A-N from the data store 138. The determiner 128 can use such models 142A-N to evaluate the condition of the MCSD 104. For example, as described throughout this disclosure, the determiner 128 can determine whether the MCSD 104 is operating normally or abnormally. The MCSD 104 can determine what abnormal condition or conditions the MCSD 104 is experiencing, such as pump thrombosis. The determiner 128 can also determine whether the MCSD 104 is likely to develop abnormal conditions such as pump thrombosis.

The output generation engine 130 can be configured to generate output about the condition of the MCSD 104, as determined by the device condition determiner 128. For example, the engine 130 can generate a notification, text message, visual indication or representation, and/or alert indicating the condition of the MCSD 104. The engine 130 can determine what type of output to generate based on a severity of the condition. For example, if the device condition determiner 128 determines that the MCSD 104 has pump thrombosis or other mechanical malfunction, the output generation engine 130 can generate and transmit an alert to a user device of a clinician that instructs the clinician to replace the pump immediately. As another example, if the determiner 128 determines that the MCSD 104 is operating normally, the engine 130 may generate a notification that tells the clinician that everything is okay and the MCSD 104 does not need to be inspected at a present time. One or more other outputs can be generated by the engine 130 and transmitted to one or more other devices and/or systems, as described throughout this disclosure.

The models training and generation engine 132 can be configured to train the device condition model(s) 142A-N. In some implementations, the models training and generation engine 132 can be part of a separate computer, device, system, cloud-based service, and/or network of computers, devices, or systems. The engine 132 can train the models 142A-N to process new input signals (e.g., the signals detected by the accelerometer 106, the sensor(s) 108A-N, and/or the device 136) and automatically classify the new input signals as corresponding to normal or abnormal conditions (e.g., whether pump thrombosis is present or not, or whether pump thrombosis is likely to develop or not, etc.) in the MCSD 104. The models 142A-N can be trained for each type of MCSD 104. For example, some MCSDs may produce signals of a first frequency that are indicative of abnormal operational conditions while other MCSDs may produce signals of a different, second frequency that are indicative of abnormal operational conditions. During run time, the device condition determiner 128 can select the model or models 142A-N that were trained to analyze signals produced by the particular MCSD being evaluated by the computer system 102. In some implementations, the models 142A-N can be generic and trained to evaluate signals produced by all types of MCSDs. In yet some implementations, one or more of the models 142A-N can be trained to evaluate signals produced by specific categories and/or groupings of MCSDs. The models 142A-N that are generated and trained by the engine 132 can be stored in the data store 138 and accessible by the device condition determiner 128 during run time.

The models training and generation engine 132 may also continuously improve the models 144A-N based on evaluations that are made by the device condition determiner 128 over time. The engine 132 may also improve the models 144A-N to more accurately evaluate signals based on user input that is received by the input device(s) 110 (e.g., a manual evaluation of the MCSD 104's pump by the clinician, which reveals pump thrombosis; manual review of acoustic recordings and labeling of frequencies by the clinician, etc.).

The communication interface 134 can provide for communication between one or more of the components described in FIG. 1B.

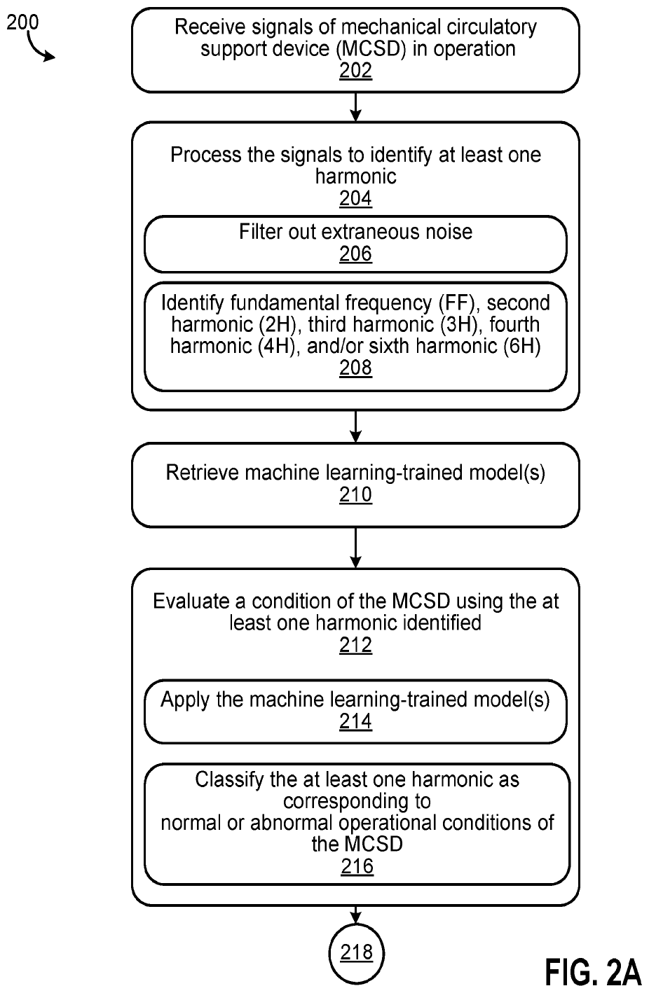

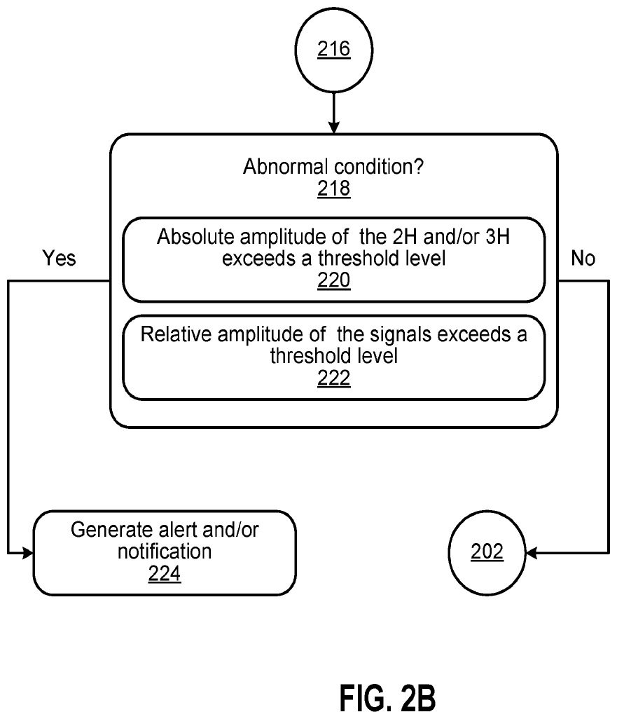

FIGS. 2A-B is a flowchart of a process 200 for evaluating an operational condition of a MCSD. The process 200 can be performed by the computer system 102. In some implementations, one or more steps in the process 200 can also be performed by the condition surface detection device 136, depicted and described in FIG. 1B. The process 200 can also be performed by one or more other devices, computers, systems, cloud-based services, and/or networks of computers or devices. For illustrative purposes, the process 200 is described from a perspective of a computer system.

Referring to the process 200 in both FIGS. 2A-B, the computer system receives signals of the MCSD in operation in 202. A sensing subsystem can be configured to obtain from one or more sensors signals indicative of at least one of vibrations, acoustics, or electromagnetic signals of the MCSD in operation in the recipient (e.g., a mammal, such as a human or animal). The signals indicative of at least one of the vibrations, acoustics, or electromagnetic signals of the MCSD in operation are transmitted from the sensing subsystem to the computer system.

In 204, the computer system processes the signals to identify at least one harmonic in the signals. Processing the signals can include filtering out extraneous noise (206). Processing can also include identifying FF, 2H, 3H, 4H, 6H and potentially higher order harmonics in the signals (208). For example, processing the signals can include aurally identifying FF, 2H, and 3H as audible tones, reproducing the audible tones into generated tones using a signal generator, superimposing the generated tones into a sound recording, fine tuning frequency of the generated tones in the sound recording by identifying beats, identifying visual waveforms based on playing back the sound recording, performing FFT on the sound recording, labeling frequencies in the sound recording, and labeling higher order harmonics that are inaudible but are identified based on performing the FFT.

The computer system can retrieve machine learning-trained models in 210. The computer system can retrieve models that are trained to evaluate conditions of the particular MCSD. The computer system can retrieve generic models that are trained to evaluate conditions of any type of MCSD or a category or group of MCSDs. The computer system can also retrieve models that are trained to identify particular types of conditions in any type of MCSD or the particular MCSD. Block 210 can be performed at any time before or during blocks 202-208.

In 212, the computer system evaluates a condition of the MCSD using the at least one harmonic that was identified in block 204. To evaluate the condition of the MCSD, the computer system applies the model(s) in 214. The computer system then classifies the at least one harmonic as corresponding to normal or abnormal operational conditions of the MCSD (216). Classifications of the MCSD conditions can be output from the applied model(s). For example, the at least one harmonic can be provided as input to the model(s). The model(s) can then output an indication of whether the MCSD has normal or abnormal operational conditions based on analyzing the at least one harmonic. As described above, the model(s) can be trained using training datasets that include signals labeled with normal operational conditions and signals labeled with abnormal operational conditions.

The computer system then determines whether abnormal conditions were identified in 218. Abnormal operational conditions of the MCSD include at least one of presence of pump thrombosis and likelihood to develop pump thrombosis at the MCSD. Abnormal conditions may exist where an absolute amplitude of the 2H and/or 3H exceeds a threshold level (220). As described further below, higher order harmonics having frequencies within or above predetermined ranges can be indicative of pump thrombosis. Abnormal conditions may also exist where a relative amplitude of the signals exceeds a threshold level (222). The relative amplitude can be a ratio of an amplitude of the 3H to an amplitude of the FF in the signals. As mentioned above and described further below, a ratio of 3H to FF amplitude that exceeds a predetermined threshold range can be indicative of pump thrombosis.

If the abnormal conditions were identified, then the computer system generates an alert and/or notification in 224. For example, the computer system can output, at a user device, an alert notification identifying the abnormal conditions of the MCSD, such as pump thrombosis. One or more other types of output can be generated by the computer system, as described above in reference to FIGS. 1-2.

If abnormal conditions were not identified, the computer system can return to block 202 and repeat the process 200. In other words, the computer system has identified that the MCSD is operating normally at the current time of evaluation. In some implementations, the process 200 may stop after determining that abnormal conditions were not identified. The process 200 can be repeated at another time (e.g., on some schedule, at predetermined time intervals, etc.) or when a new input signal is detected and received.

### Example Implementation

The disclosed techniques can be used to characterize properties of audible tones, vibrations, and/or electromagnetic signals produced by LVAD pumps or other MCSDs in order to ascertain whether changes in those tones or signals are present in a setting of pump thrombosis. In some implementations, surface recordings can be obtained using high fidelity digital stethoscopes or other sensors. Audio data from such surface recording can be analyzed using machine learning-based digital recording and editing techniques to produce an acoustic spectrogram by Fast Fourier transformation (FFT). As described above and further below, a computer system can therefore detect whether pump thrombosis or other abnormal operational conditions exist from the analysis of audible tones, vibrations, and/or electromagnetic signals.

In an example implementation, audio recordings can be obtained in 53 patient encounters across three LVAD pump models, HeartMate II, HeartWare, and HeartMate III: 27 recordings for HeartMate II, 19 for HeartWare, and 7 for HeartMate III). The HeartMate II left ventricular assist system (LVAS) is a second generation axial flow device and the HeartWare Heart Wear Ventricular Assist Device (HVAD) and HeartMate III LVAS are third generation centrifugal flow devices. In 12 patients (9 HeartMate II and 3 HeartWare) there was clinical concern for pump thrombosis. In all patients and pump models, a fundamental frequency was identified using the techniques described herein, and second and third harmonics were also clearly identified. Where thrombosis occurred in the HeartMate II pump, absolute (e.g., normal −46.9 [−57.5,−42.9] decibels (dB) vs. thrombosis −41.4 [−49.8,−26.8] dB, p=0.08) and relative (e.g., normal 0.72 [0.62, 0.92] vs. thrombosis 0.95 [0.86, 1.24], p=0.01) third harmonic frequencies were increased in amplitude. Where paired data was available, an increase in the absolute and relative third harmonic frequencies was seen in all patients. In the case of the HeartWare device, a consistent difference in harmonic amplitudes in the setting of thrombosis was not identified, however is likely to be demonstrable in a centrifugal device such as this using a triaxial accelerometer or hydrophone for detection of higher order harmonics with a high signal to noise ratio The HeartMate 3 is an example of a centrifugal device. Accordingly, a consistent pattern of fundamental and harmonic frequencies is common to all LVADs. Alterations in the amplitude of higher order harmonics can signal the onset of pump thrombosis in axial flow LVADs or other MCSDs.

Where the absolute amplitudes of fundamental and harmonic frequencies in the setting of normal pump function and the setting of suspected thrombosis were compared, the absolute amplitude of the third harmonic frequency, and more notably the relative amplitude of the third harmonic frequency as compared with the amplitude of the fundamental, were increased on recordings from the HeartMate II axial flow pump. Where paired data were available before and after the onset of pump thrombosis, an increase in these measures were seen in all patients compared to baseline. The most consistent alteration in the setting of HeartMate II pump thrombosis is to the third harmonic frequency. As such it appears there may be a specific quality about the third harmonic that predisposes to its increase in the setting of thrombosis on the HeartMate II device. Higher order harmonics may be reliably detected in LVAD recordings utilizing more sensitive technology, as described throughout this disclosure.

Although the example implementation is described in reference to using a stethoscope to obtain audio recordings, the disclosed techniques can be performed using a triaxial accelerometer to obtain high resolution frequency recordings from LVAD recipients with and without suspected pump thrombosis. For example, a surface triaxial accelerometer or hydrophone can be placed on the chest surface in a position that obtains optimal resolution of high harmonic frequencies based on live assessment of FFT of the vibration signals. The desired position for placement can be a left lower sternal border, approximately the surface marking for auscultation of the tricuspid valve. A signal conditioner and data acquisition card can be used to allow analysis of these vibration signals through a computer system or computing device (e.g., laptop, tablet, computer, mobile phone, smartphone, remote computing system, network of computers or devices, cloud-based service), and dedicated software, program, or application for acquisition and analysis of these signals at very high sampling rates (e.g., refer to FIGS. 1-2).

Moreover, customized analysis techniques can be developed using machine learning techniques specifically adapted to non-linear high frequency harmonic signals, likely to be present in this frequency range in the setting of pump thrombosis. Harmonic frequencies obtained from LVADs are non-linear, and machine-learning based techniques can be used to consistently and accurately analyze very high frequency harmonic signals. The machine-learning based techniques can provide a quantitative comparison of waveforms and their changes in the setting of pump thrombosis in order to consistently and accurately detect pump thrombosis.

Finally, analysis of very high frequency harmonic signals using high frequency sampling as described throughout this disclosure can yield granular data that may be applicable to development, training, and continuous improvement of deep learning algorithms and/or models used for early detection of pump thrombosis.

Although the example implementation is described with regards to analysis of acoustic signals/recordings, the disclosed technology can also provide for consistent detection of higher order frequencies with a high signal to noise ratio. This can require a shift from detection of audio signals along to also include detection of higher frequency vibration signals and/or electromagnetic signals generated by the LVAD itself. Therefore, the disclosed techniques can also provide for analysis of high frequency vibration and electromagnetic signals detected and echocardiography and diagnostic ECG in patients who have developed LVAD pump thrombosis.

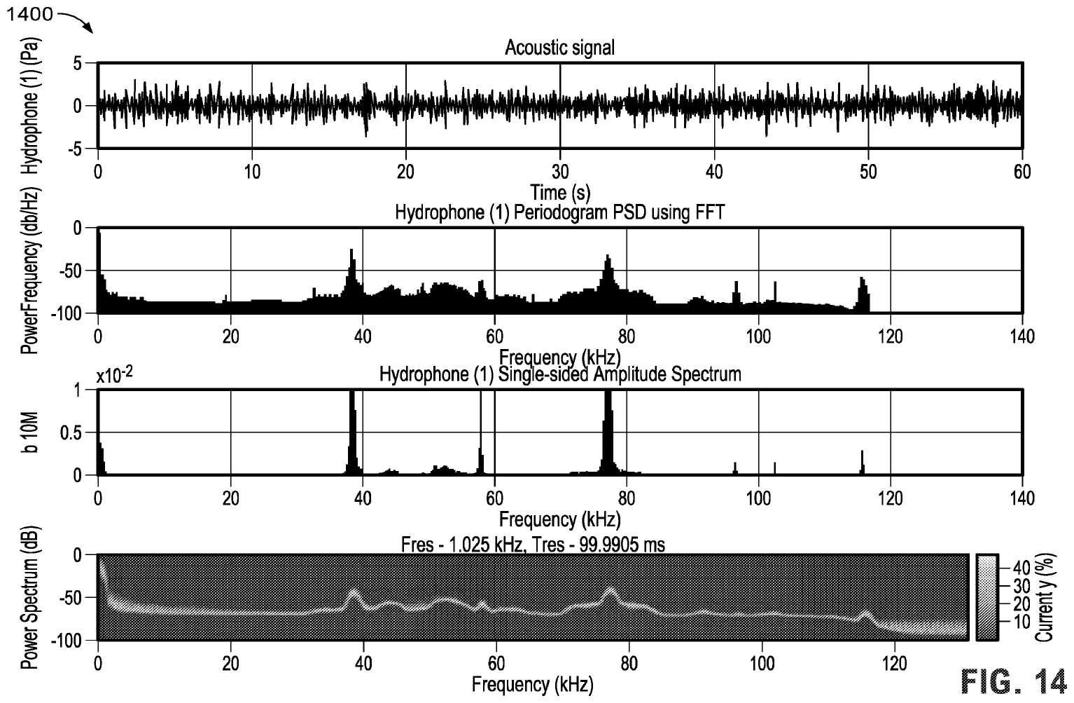

Accordingly, the disclosed techniques can be implemented in a device that detects high frequency vibration signals created by an LVAD. Vibration signals can, for example, be detected directly on the LVAD. Vibration signals can also be detected using a handheld detection device or system. The detection device or system can include a triaxial accelerometer or hydrophone, one or more (e.g., 2) signal conditioners, a data acquisition system (e.g., 16 bit data acquisition system), and a computer system or device with installed signal acquisition software. Refer to FIGS. 14-19 for further discussion.

Referring to the figures, FIG. 3 illustrates a process 300 for identifying frequencies from an LVAD sound recording. The process 300 can be performed by a computer system, such as the computer system 102 described in reference to FIG. 1. The process 300 can also be performed by one or more other computer systems, devices, networks of computers, and/or cloud-based services.

Before the process 300 begins, a digital stethoscope or other sensor(s) can be used to obtain sound recordings. More particularly, surface audio recordings can be obtained in the second right intercostal space (aortic area), second left intercostal space (pulmonary area), fourth left intercostal space at the sternal border (tricuspid area), and fifth ICS, left mid-clavicular line (mitral area) of patients who have undergone LVAD implantations. Duration of each recording in each position can be 30 seconds. One or more other recording durations can also be used, including but not limited to 15 seconds, 20 seconds, 25 seconds, 35 seconds, 40 seconds, etc. A filter on the stethoscope can be set for a flat equalization (EQ) so that all frequencies can be detected equally and without suppression of higher frequencies, which may occur by design in standard clinical electronic stethoscopes. The sampling rate can be 44 kHz for an audio based system but much greater for a triaxial accelerometer or hydrophone.

The recordings can then be analyzed using machine learning-based digital audio recording and editing software/applications. The signals can be trimmed and extraneous noise can be removed. No low or high pass filters may be applied. Predominant frequencies heard by ear (e.g., via aural assessment) can be matched using a signal generator and oscilloscope. An acoustic spectrogram can then be obtained using a FFT.

Frequencies and their amplitudes on the acoustic spectrogram can be identified, and characterized as fundamental frequency, or harmonics, based on initial aural assessment. Relative amplitudes can be compared to the fundamental frequency and expressed as a ratio. The relative amplitude can be calculated as (Fundamental Amplitude/Absolute Amplitude). Waveforms in each sound recording can also be analyzed qualitatively.

Finally, suspected LVAD pump thrombosis can be defined as an acute rise in serum lactate dehydrogenase (LDH) ≥2-fold above stable baseline levels, with evidence of intravascular hemolysis (elevated plasma free hemoglobin, reduced haptoglobin or hemoglobinuria) and either new impairment of LV offloading or power surges ≥2 W above baseline.

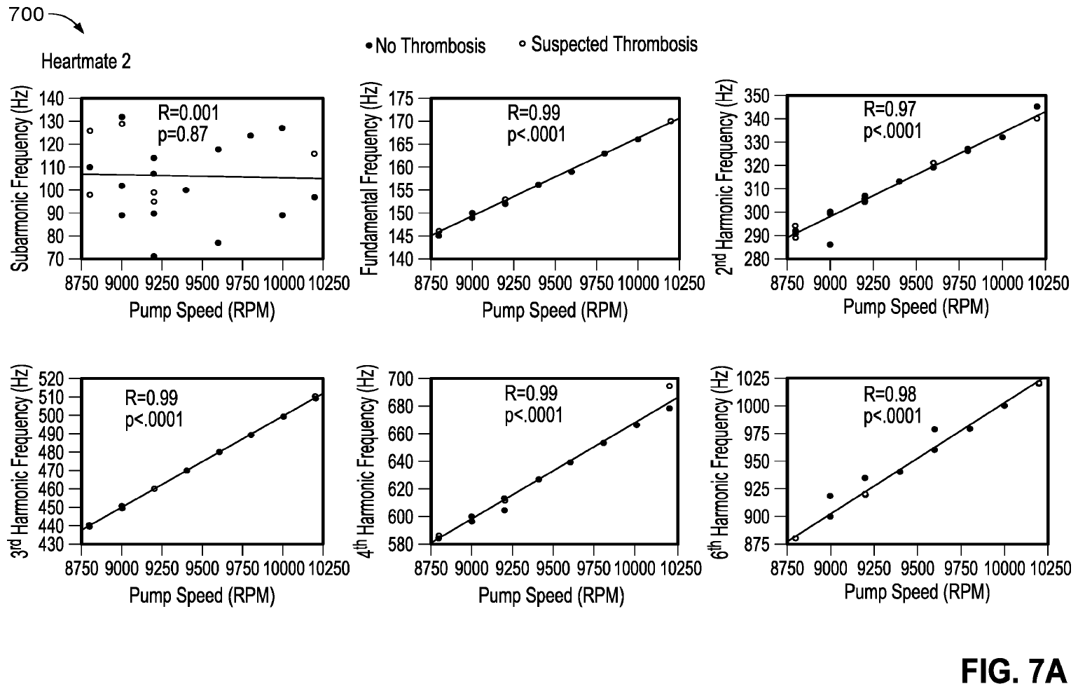

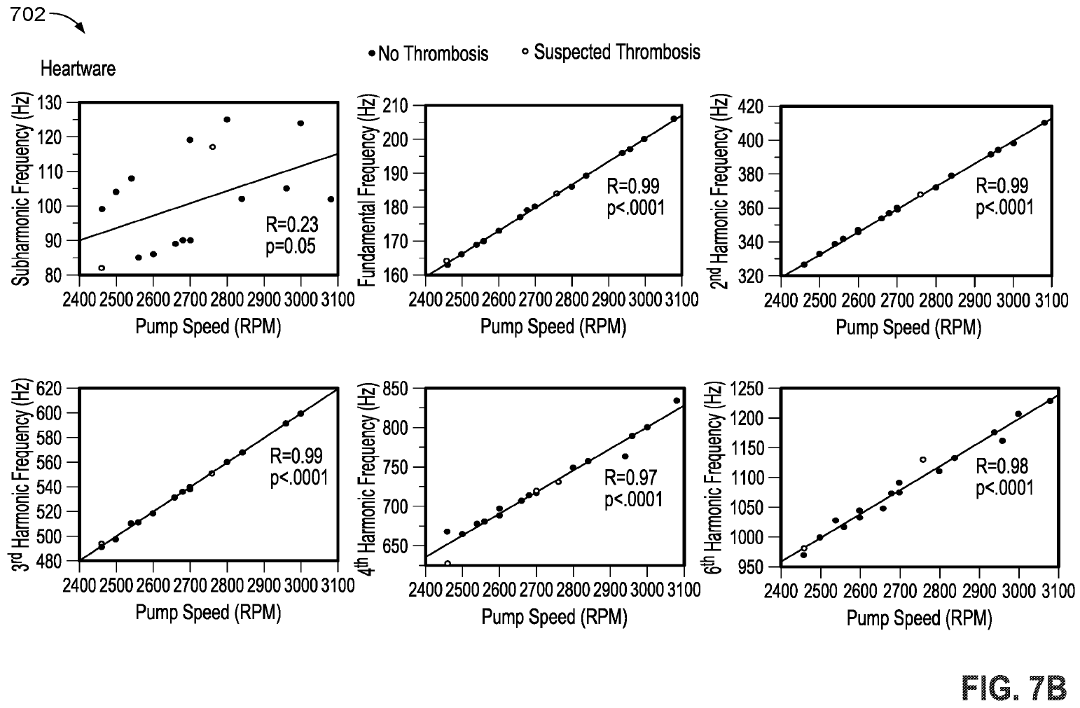

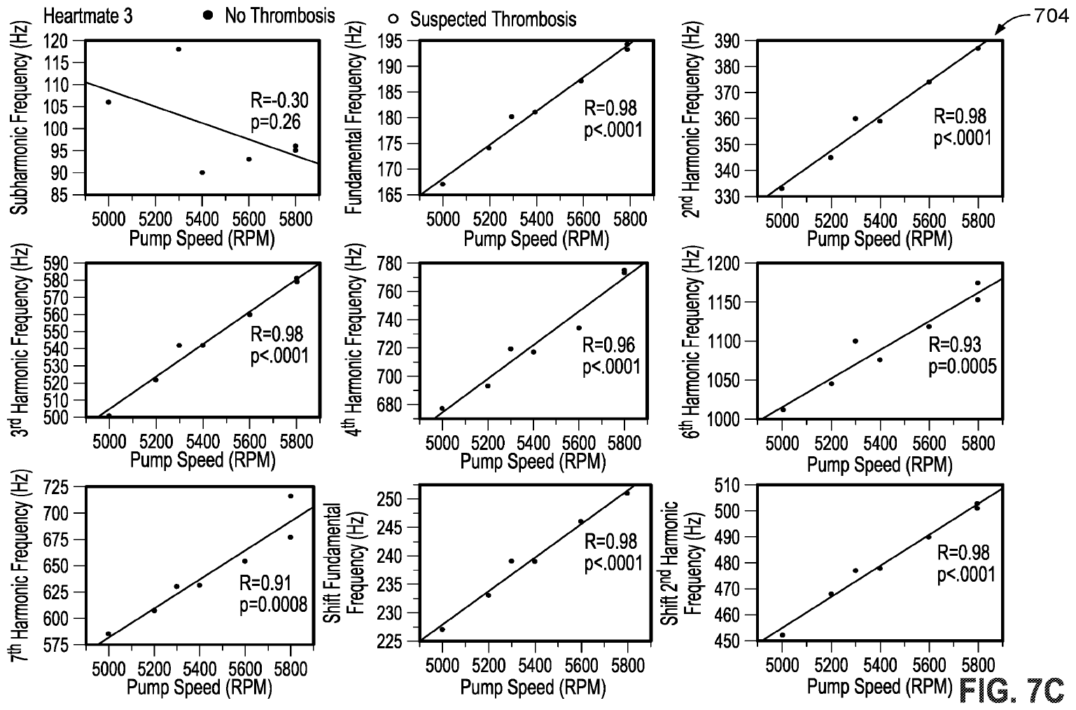

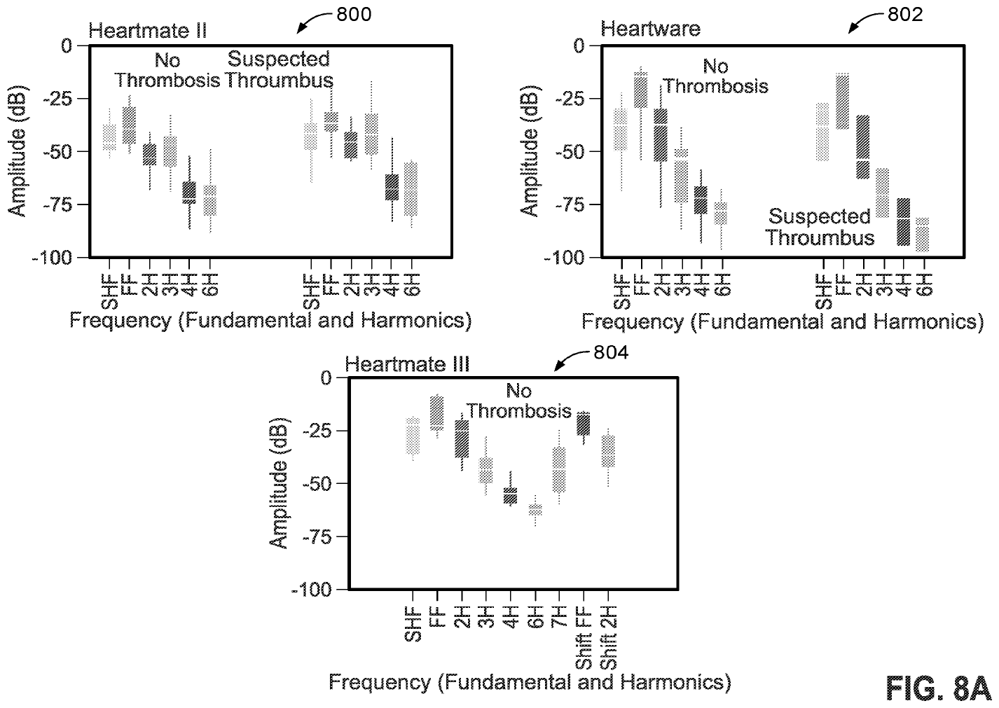

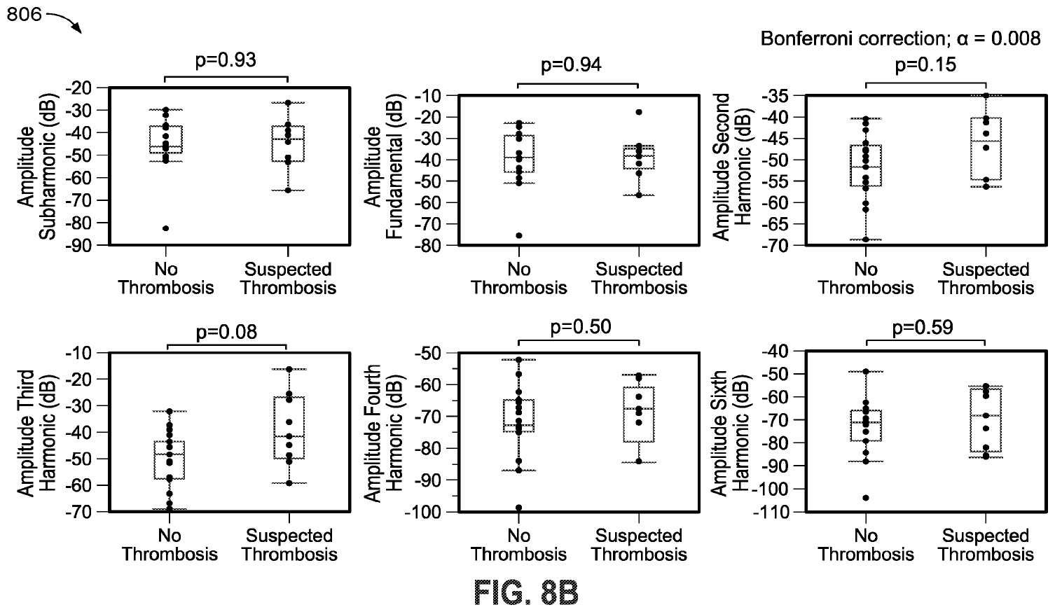

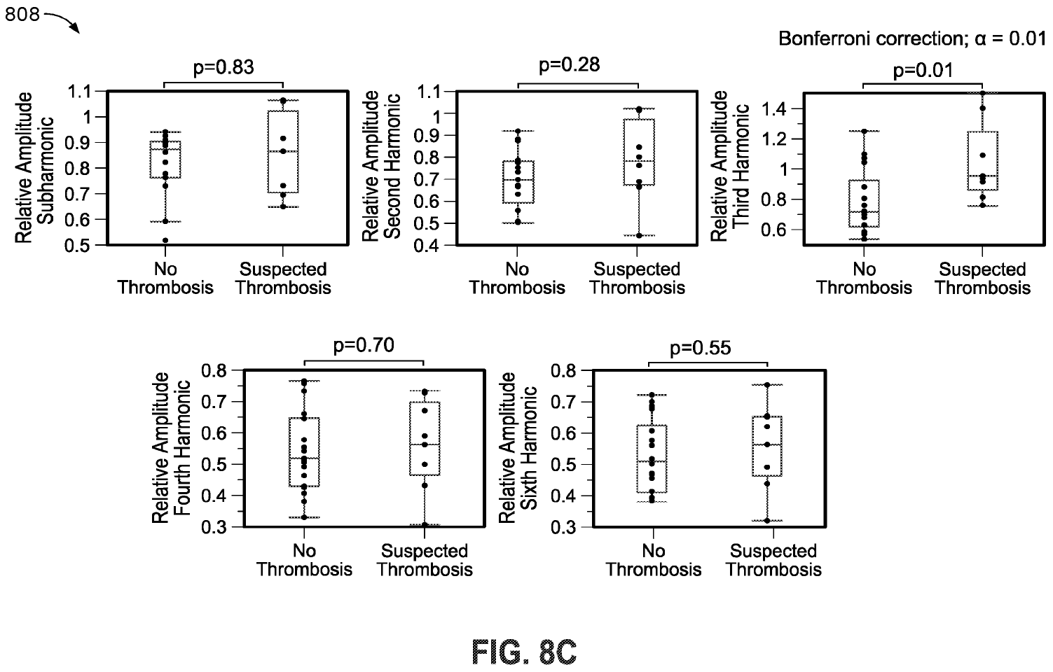

The sound recordings can be played back and tones can be aurally identified in 302. Usually, one to three tones can be distinguished, which includes fundamental frequency (FF), second harmonic (2H), and third harmonic (3H) frequencies. Occasionally, higher order frequencies, such as fourth harmonic (4H) and sixth harmonic (6H) can be distinguished as well at much lower amplitudes, close to a level of background noise. A subharmonic frequency (SHF) may also be identified in all devices indicative of presence of non-linearities in mechanical vibrations produced in LVADs. The FFs and all harmonic frequencies in all device types, with the exception of the SHFs, can be linearly related to the pump speed without consistent deviation in presence of thrombosis {R=0.95-0.99, p<0.001} (e.g., refer to FIGS. 7-8).

In 304, audio tones, which usually includes FF, 2H, and 3H, can be reproduced using a signal generator. Next, in 306, sound recordings and generated tones can be superimposed. The generated tones frequency can be fine-tuned by identifying beats. In 308, FF, 2H, and 3H frequencies can be identified and frequency values can be confirmed.

The sound recording can now be played back and visual waveforms can be noted using analysis software, applications, or programs (310). FFT can also be performed on the sound recording in 312. After FFT, frequencies can be labelled based on the aural identification process in 302 and sub and higher order harmonics (e.g., inaudible but seen on FFT) can also be labelled (314).

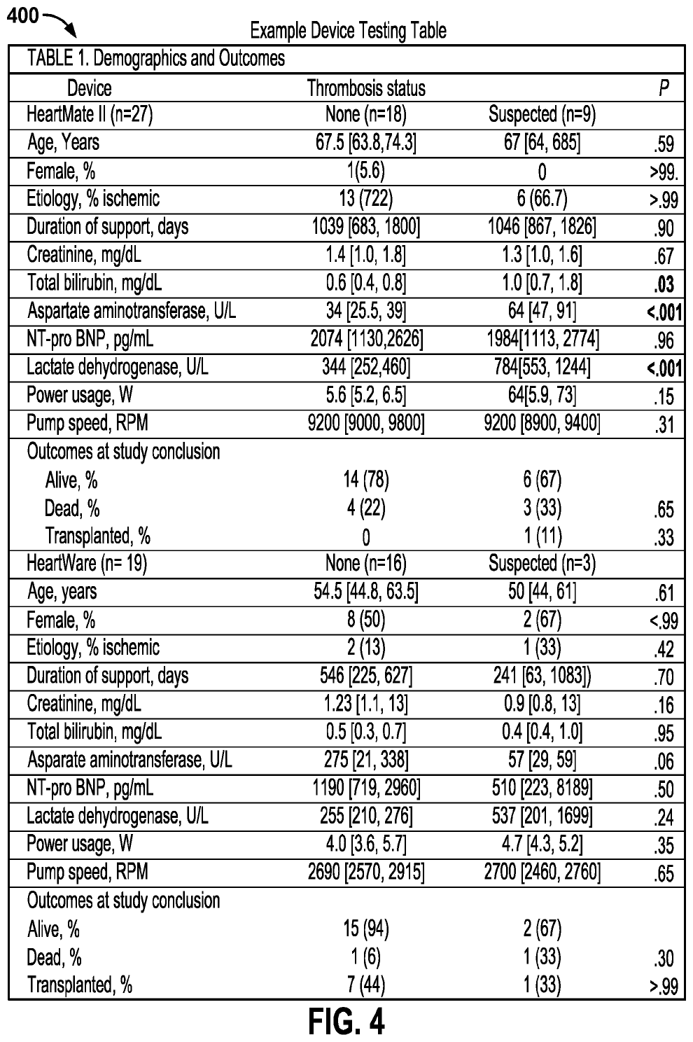

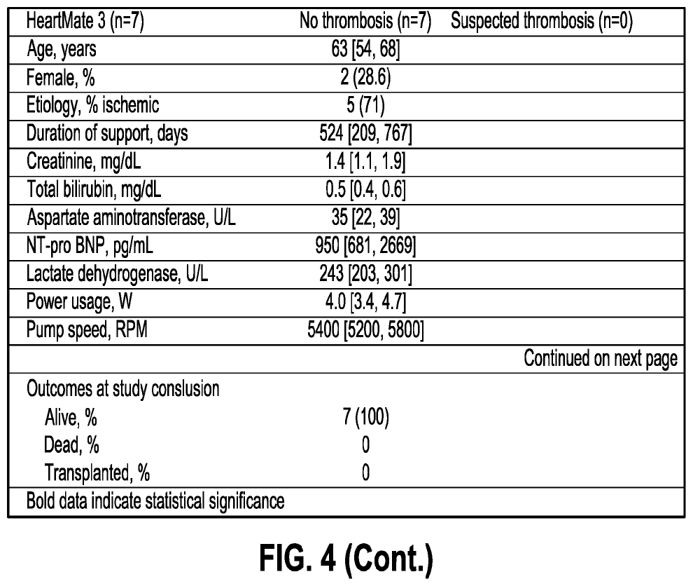

FIG. 4 depicts a table 400 demonstrating differences among patients with and without thrombosis with different exemplary LVAD types. In this example implementation, sound recordings can be obtained from 47 patients: 22 HeartMate II, 18 HeartWare, and 7 HeartMate III. Paired data before, and after onset of thrombosis, can be obtained in 5 HeartMate II recipients and 1 HeartWare recipient, thus a total of 27 recording encounters can be accrued for the HeartMate II device and 19 for the HeartWare device. The presenting characteristics and laboratory values of each group at time of recording as well as subsequent outcomes are summarized in the table 400. 4 recordings were obtained during each encounter (aortic, pulmonary, tricuspid, and mitral areas). Highest quality recordings and most consistent distribution of frequencies were obtained at the tricuspid area, both on aural assessment and analysis of spectrograms, and only these recordings were used for further analysis in the example implementation of the disclosed techniques.

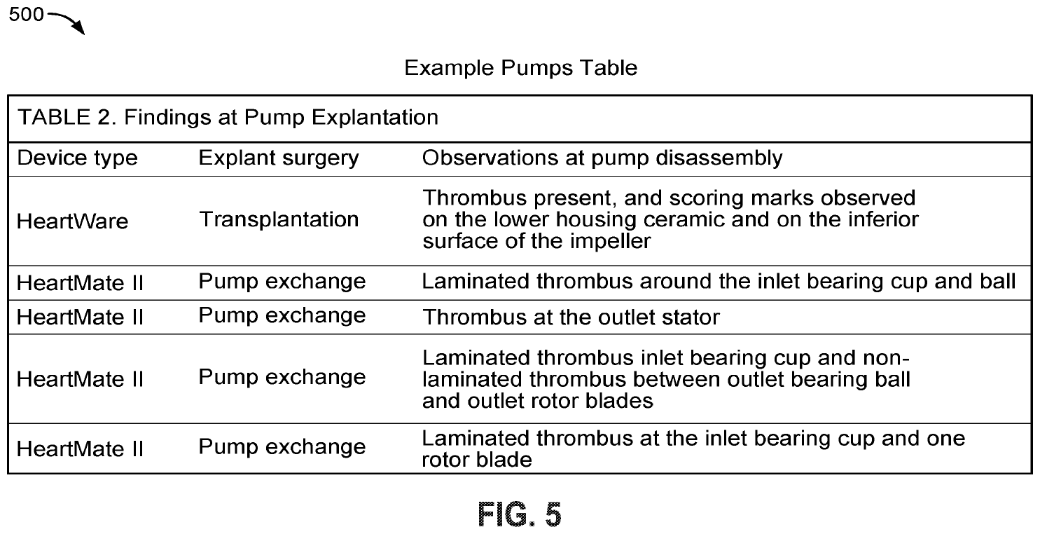

FIG. 5 depicts a table 500 describing exemplary findings at pump disassembly where thrombosis was suspected. Referring to both FIGS. 4 and 5, In 12 patients (9 HeartMate II, 3 HeartWare) there was a clinical concern for pump thrombosis. There were no thrombotic events in the 7 HeartMate III patients. Of those 12 patients with suspected pump thrombosis, 7 pumps were explanted (5 at pump exchange, 2 at transplantation) and 5 were managed conservatively with intravenous unfractionated heparin and tirofiban. A total of 4 patients with suspected pump thrombosis died, 3 following pump exchange, and 1 after transplantation. Among the 7 patients who had pumps explanted, where a report was obtained following disassembly of the pump, thrombus was present in all (n=5). The location of thrombus seen is shown in the example pumps table 500 in FIG. 5.

Suspected thrombotic events that occurred were defined by an acute rise in serum LDH ≥2-fold above stable baseline levels, with evidence of intravascular hemolysis (elevated plasma free hemoglobin, reduced haptoglobin or hemoglobinuria) and either new impairment of LV offloading or power surges ≥2 W above baseline. Where thrombosis was suspected and devices explanted and analyzed, thrombus was detected in 100% of such devices.

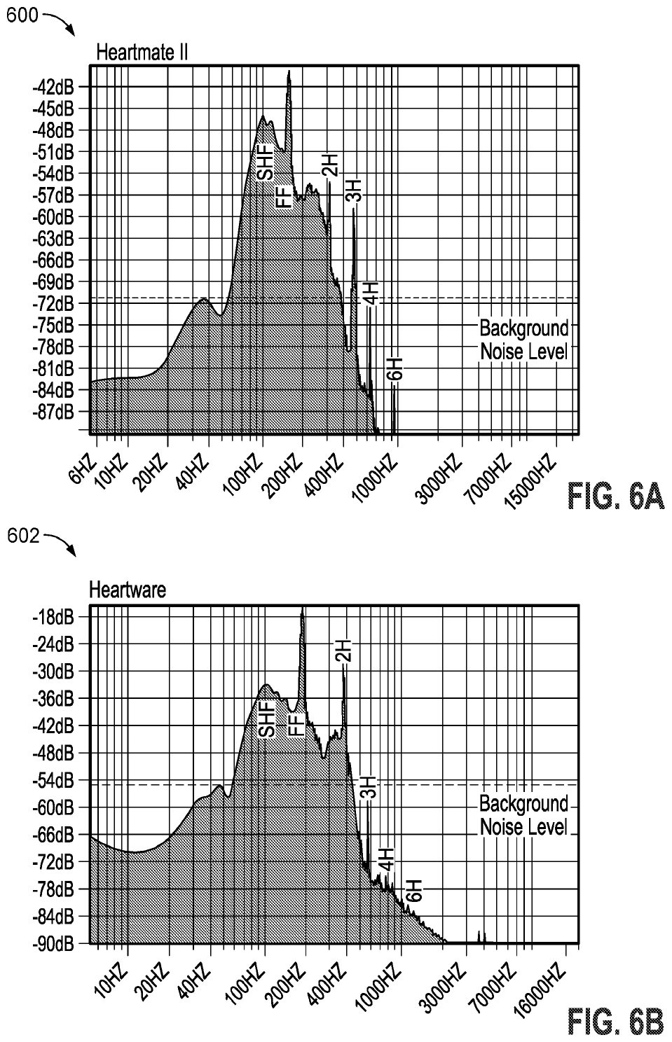

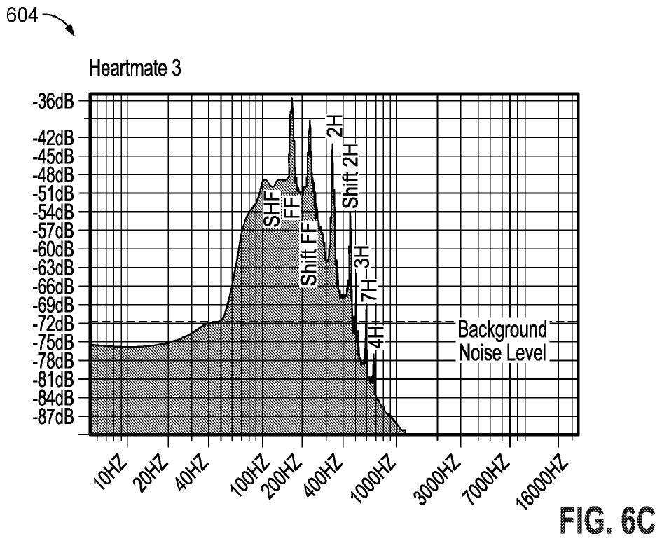

FIG. 6 depicts graphical representations 600, 602, and 604 of Fast Fourier transformations on sound recordings for three example LVADs, HeartMate II, HeartWare, and HeartMate III, respectively. In all patients (HeartMate II, HeartMate III and HeartWare) in the example implementation, FF is identified, and 2H and 3H are clearly detected. 4H and 6H can also be detected in all recordings but may appear lower in amplitude, close to a level of background noise. A subharmonic frequency (SHF) can also be identified in all devices, as shown and labeled in the graphs 600, 602, and 604.

In the case of the HeartMate III device, a distinct seventh harmonic (7H) was audible, which was not detectable in the HeartMate II and Heart Ware devices. Refer to the graph 604. The HeartMate III also has a periodic programmed ramp-up in pump speed, which for the purposes of this example implementation is termed a “shift frequency” (Shift F). On an FFT, the Shift F is detected as a higher frequency spike, which is 5 semitones (or a musical perfect fourth interval) above the FF. The Shift F has itself its own fundamental frequency (ShiftFF) and harmonics (for simplicity only the second harmonic (Shift2H) is analyzed and depicted in the graph 604).

FIGS. 7A-C depict graphical representations of Spearman rank correlations between pump seeds and frequencies for each pump type in three example LVADs. As shown in FIGS. 7A-C, fundamental frequencies and all harmonic frequencies in all device types, with the exception of the subharmonic frequencies, are linearly related to the pump speed. Assessment of agreement with the predicted frequency for each pump speed did not yield a significant difference in the presence or absence of thrombosis. FIG. 7A depicts graphical correlations 700 between pump speeds and subharmonic frequency, FF, 2H, 3H, 4H, and 6H for the HeartMate II device. FIG. 7B depicts graphical correlations 702 between pump speeds and subharmonic frequency, FF, 2H, 3H, 4H, and 6H for the HeartWare device. FIG. 7C depicts graphical correlations 704 between pump speeds and subharmonic frequency, FF, 2H, 3H, 4H, 6H, 7H, ShiftFF, and Shift2H for the HeartMate III device. As shown in FIG. 7C, 7H, ShiftFF, and Shift2H are also linearly related to pump speed.

FIG. 8A depicts graphical representations of amplitudes of frequencies by pump type and suspicion for thrombosis. More particularly, distributions of amplitudes by fundamental, harmonic, and subharmonic frequencies re displayed as quartile boxplots. The findings without thrombosis (left) and where thrombosis was suspected (right) are shown for each pump tight: HeartMate II LVAS 800, HeartWare HVAD 802, and HeartMate III LVAS 804. In the case of all the pump devices, FF is the loudest frequency detectable, followed by 2H and 3H. 4H and 6H are much lower in amplitude but could still be detected in all three device types. The distributions without and with suspected thrombosis are shown in FIG. 8A.

FIG. 8B depicts graphical representations comparing absolute amplitudes of frequencies where thrombosis is present or absent. Absolute amplitudes of fundamental, harmonic and subharmonic frequencies in the setting of normal HeartMate II LVAS operation (left) and suspected pump thrombosis (right) are depicted in plots 806. Comparisons between groups by Wilcoxon rank-sum test for each frequency are also depicted in the plots 806. A Bonferroni correction is performed given multiple comparisons, and the level of significance (α) is set at 0.05/6=0.008. In the setting of suspected HeartMate II pump thrombosis, the absolute amplitude of 3H frequencies trended higher compared to when thrombosis was absent (no thrombosis −46.9 [−57.5,−42.9] dB vs. thrombosis −41.4 [−49.8,−26.8] dB, p=0.08).

FIG. 8C depicts graphical representations comparing relative amplitudes of frequencies where thrombosis is present or absent. Relative amplitudes calculated as fundamental amplitude/harmonic amplitude for harmonic and subharmonic frequencies in the setting of normal HeartMate II LVAS operation (left) and suspected pump thrombosis (right) are depicted in plots 808. Comparisons between groups by Wilcoxon rank-sum test for each frequency are shown. A Bonferroni correction is performed given multiple comparisons, and the level of significance (a) is set at 0.05/5=0.01. Where the relative amplitude of 3H frequencies is calculated as a ratio of the fundamental amplitude, this is noted to be more markedly increased (no thrombosis 0.72 [0.62, 0.92] vs. thrombosis 0.95 [0.86, 1.24], p=0.01).

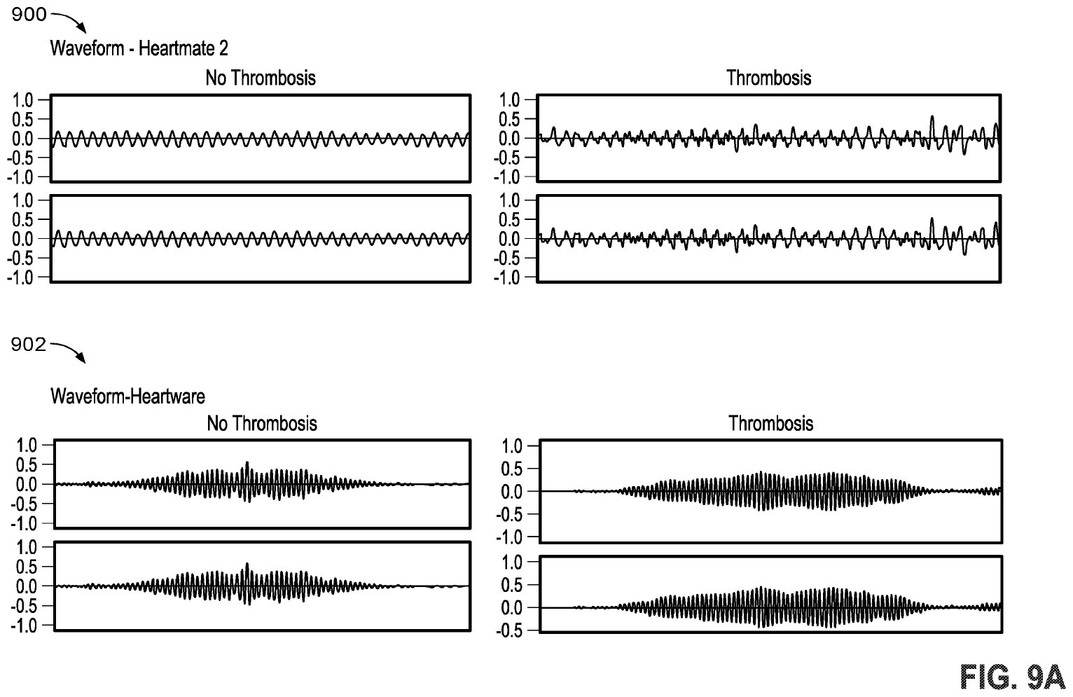

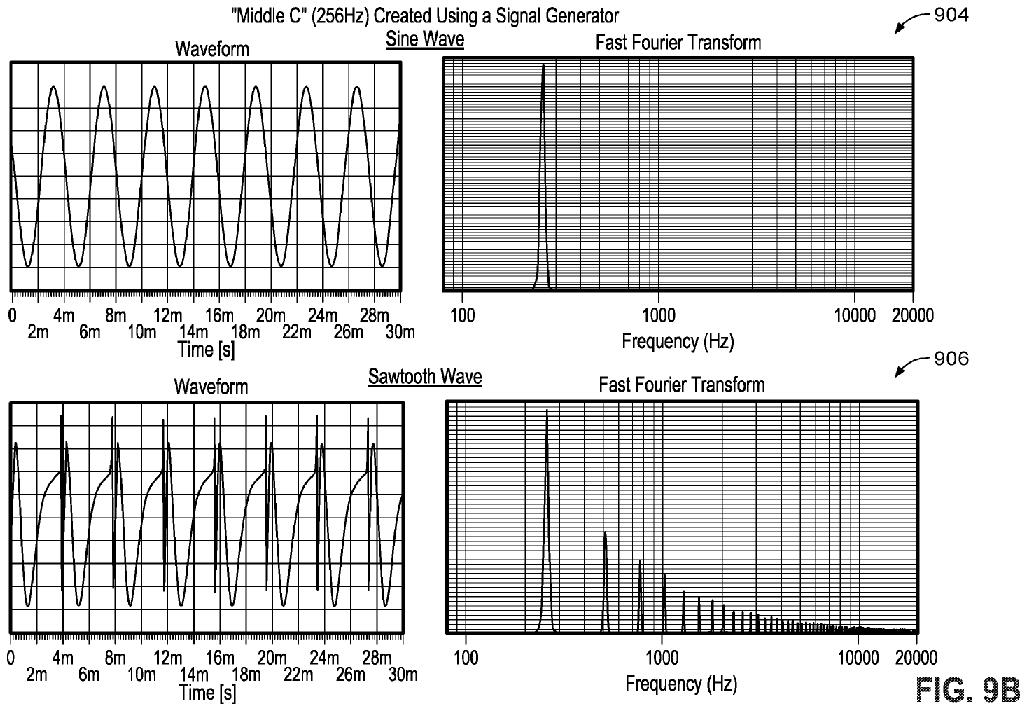

FIG. 9A illustrates visual analysis of waveforms in example LVADs where thrombosis is present or absent. Typical waveforms 900 and 902 from the HeartMate II LVAS compared to the HeartWare HVAD, respectively, are depicted in FIG. 9A. Patterns seen in the setting of suspected thrombosis are shown on the right. The Heartmate II waveform 900 amplitude is constant throughout a cardiac cycle, and in the setting of thrombosis, there is a transition from a sine to a more sawtooth waveform. The HeartWare device is notable for discrete envelopes of sound that increase in amplitude with systole, and with a less apparent visual change in the waveforms 902 where thrombosis is suspected.

Accordingly, where thrombosis is clinically suspected, the acoustic quality of the tones is often noted to be harsher on auscultation. Visual analysis of the waveforms 900 and 902 demonstrated predominantly sine waves, but with some repeating components and in the setting of thrombosis (HeartMate II), a more sawtooth pattern on qualitative assessment. This was less readily apparent to visual assessment in the case of the HeartWare, where the sound is more pulsatile and waveforms are present in discrete systolic packets of sound.

FIG. 9B illustrates Fast Fourier transformations of sine and sawtooth waves 904 and 906 respectively. The physics of a transition from a sine waveform to a sawtooth waveform is demonstrated in this figure. A pure sine wave 904 consists only of a FF, without any harmonic overlay, and demonstrated on the FFT on the right. However, if the waveform 904 is changed to a sawtooth waveform 906, multiple harmonics are detected on the FFT waveform, with diminishing amplitude as harmonic frequencies increase in value.

The timbre or smoothness/harshness of an audible tone is related to the distribution of higher order harmonics present, thus altering the waveform. The Fourier series dictates that any continuous function can be interpreted as the sum of an infinite series of sine functions. Therefore, in comparison to a sine wave (smooth sound) 904, a sawtooth waveform (harsher sound) 906 is produced by superimposition of additional sine waves at higher harmonic frequencies. Based on this rationale, the amplitude of higher frequencies would be higher in the setting of thrombosis than observed otherwise.

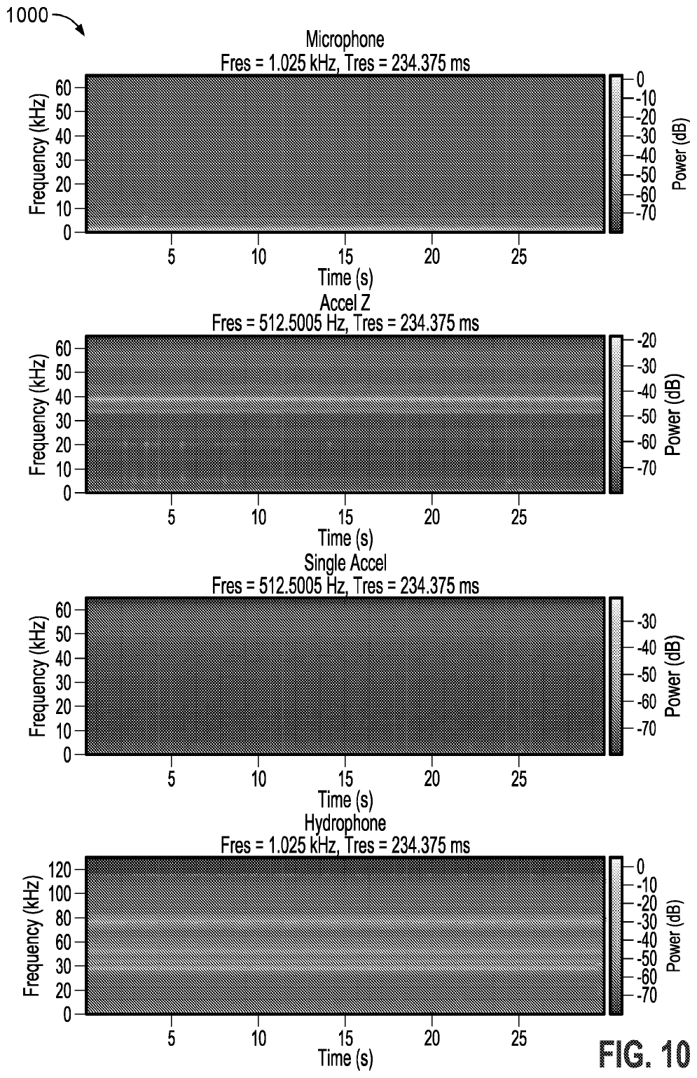

FIG. 10 is a high frequency color map 1000 comparing an electronic stethoscope (labeled “microphone”), Z-axis component of a triaxial accelerometer, uniaxial accelerometer, and hydrophone. In this example, sensors were placed on a patient's chest in the vicinity of the pump. The triaxial accelerometer was oriented such that the Z-axis was aligned with the anteroposterior axis, the X-axis was aligned with the craniocaudal axis, and the Y-axis was aligned with the mediolateral axis. The uniaxial accelerometer was aligned with the craniocaudal (X) axis. Color map 1000 depicts frequency versus time analyzed. The color map 1000 demonstrates that the Z-axis component of a triaxial accelerometer is comparable to a hydrophone in identifying high frequencies of interest. The high frequencies of interest include, in the example implementation, 20 kHz and 28 kHz for the HeartMate II LVAS, 30 kHz and 35 kHz for the HeartWare HVAD, and multiple different frequencies for the HeartMate III LVAS.

FIG. 11 is a low frequency color map 1100 comparing an electronic stethoscope (labeled ‘microphone’), Z-axis component of a triaxial accelerometer, uniaxial accelerometer, and hydrophone. Color map 1100 demonstrates that several sensors detect content in the 4-6 kHz range.

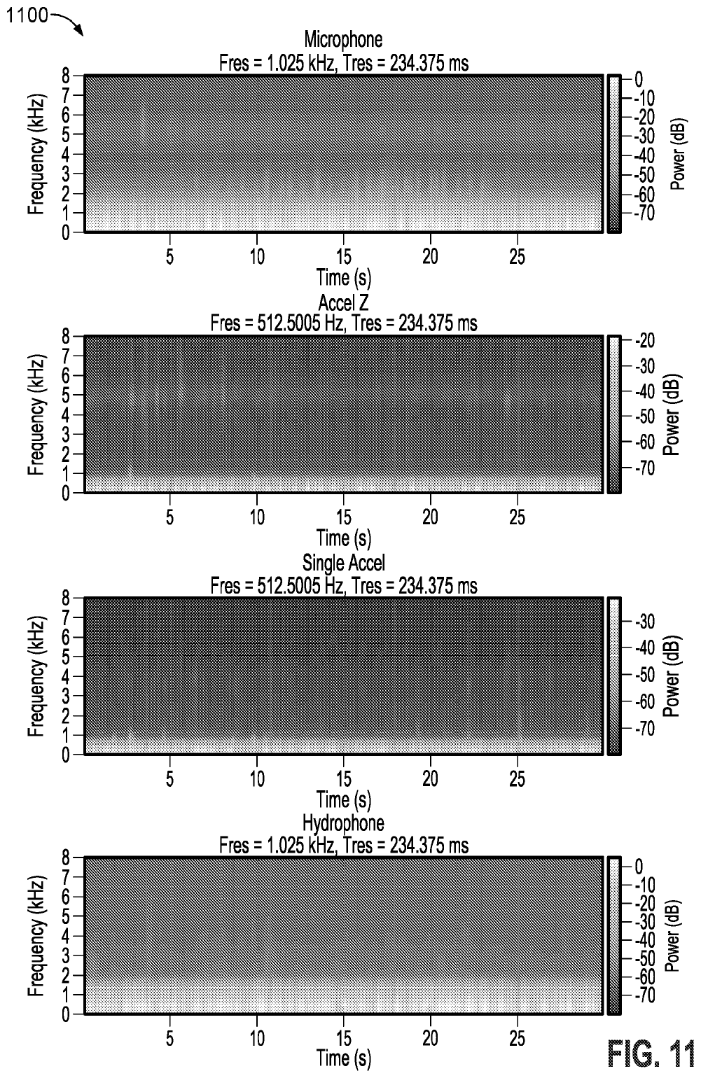

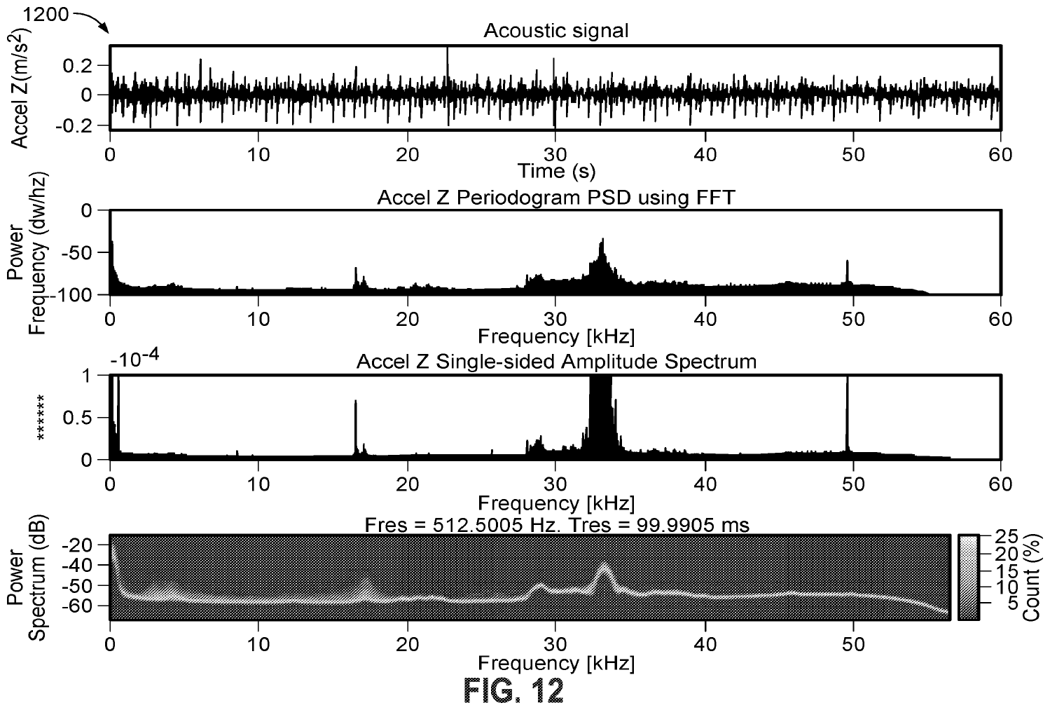

FIG. 12 depicts time frequency analysis 1200 of signals acquired from the Z-axis component of a triaxial accelerometer. The disclosed technology provides for software or other processes for analyzing frequency distribution and intensity from LVAD-derived signals, applicable to data input from various sensors, including stethoscopes, hydrophones, accelerometers, ECG, and echocardiographic data. As shown in the analysis 1200, the Z-axis component of a triaxial accelerometer provides for identification of, using the software and processes described herein, frequencies of interest in the 30 to 60 KHz band range.

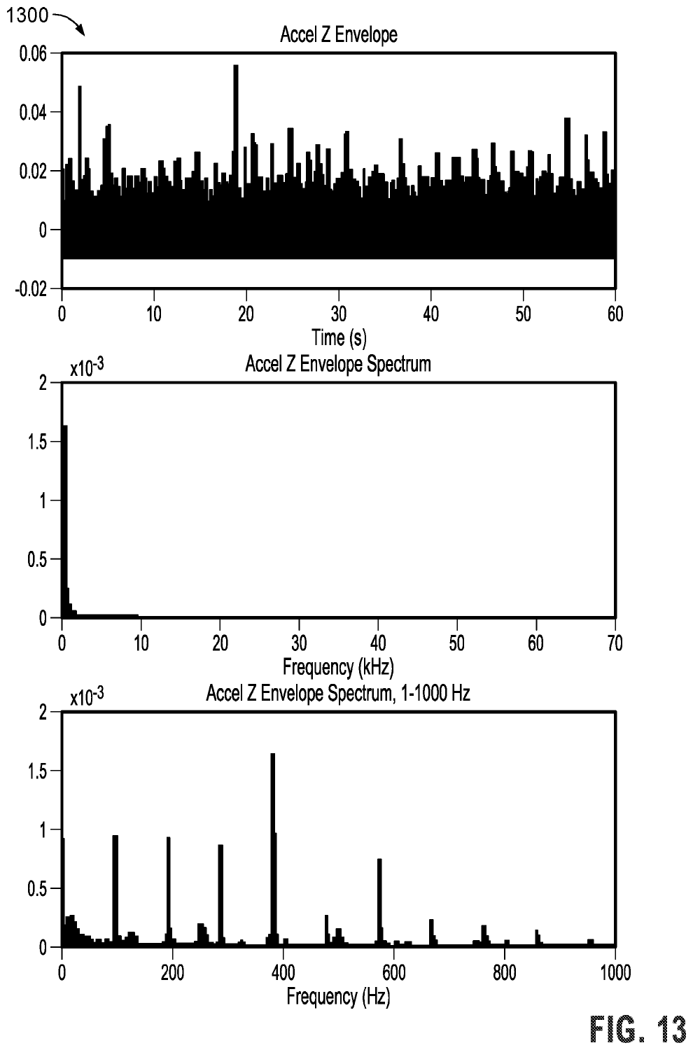

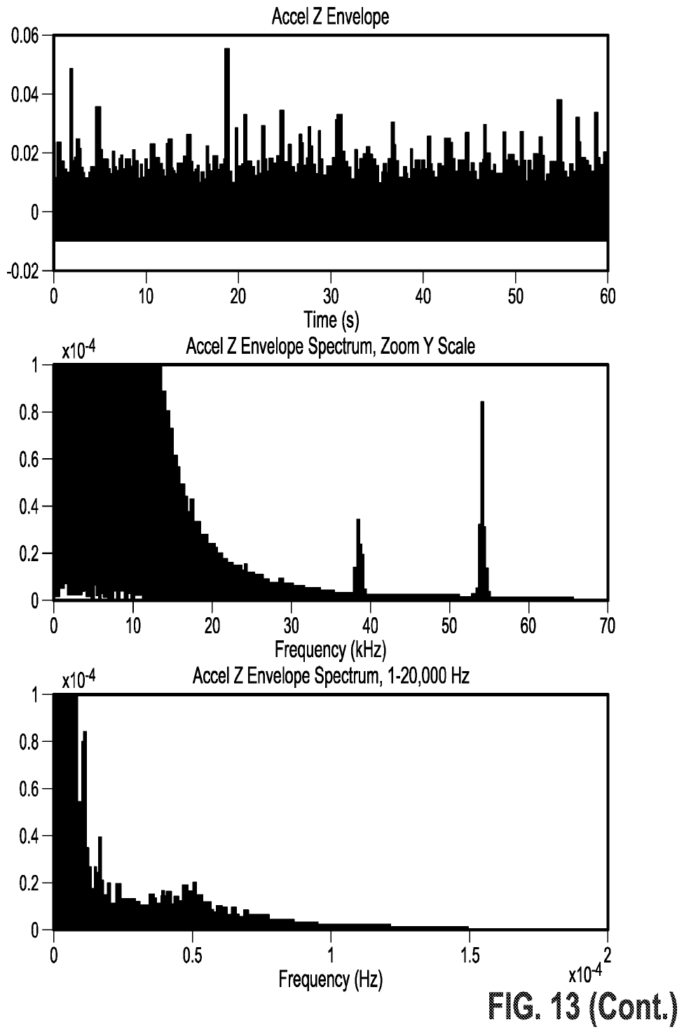

FIG. 13 depicts frequency envelope spectrum analysis 1300 of repeating signals acquired from the Z-axis component of a triaxial accelerometer. Where the analysis 1300 is performed, a higher resolution frequency analysis of repeating signals (specific to the LVAD and excluding external noise) can be obtained. As a result of the analysis 1300, a range of frequencies in the low (below 1000 Hz) and high frequency ranges can be identified, where in the case of the latter, a clear band of interest emerges above 50 kHz as well as the band already identified between 30 and 40 kHz.

FIG. 14 depicts time frequency analysis 1400 of signals acquired from a hydrophone. The hydrophone, in comparison to the triaxial accelerometer, demonstrates a more extended range of high frequency signals at 40, 60, 80 and 120 kHz from the time frequency analysis 1400.

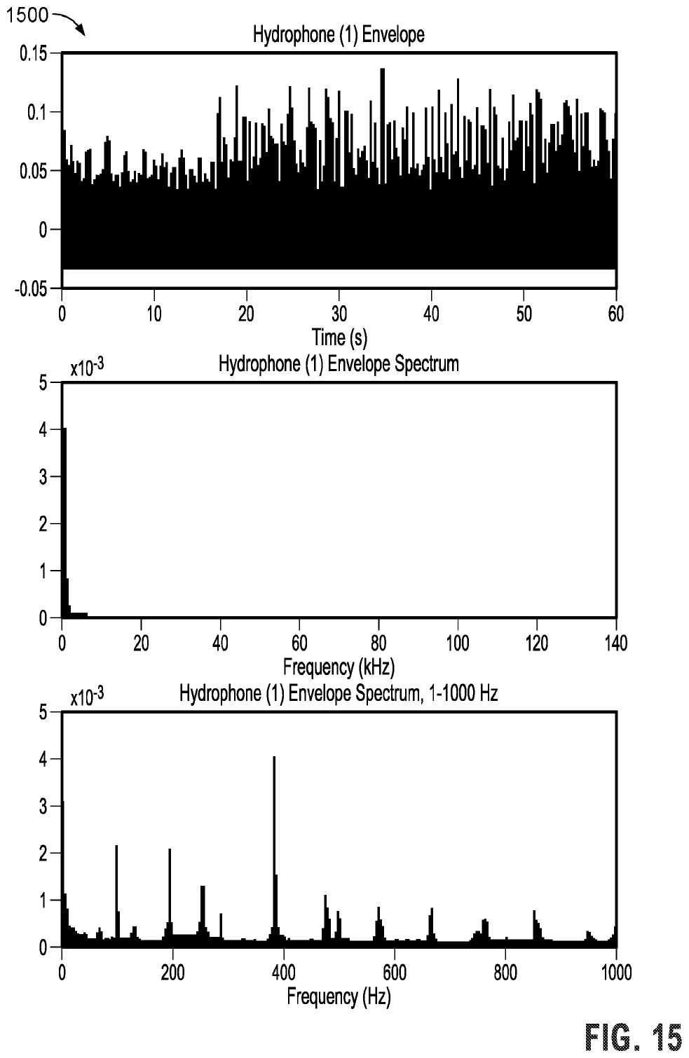

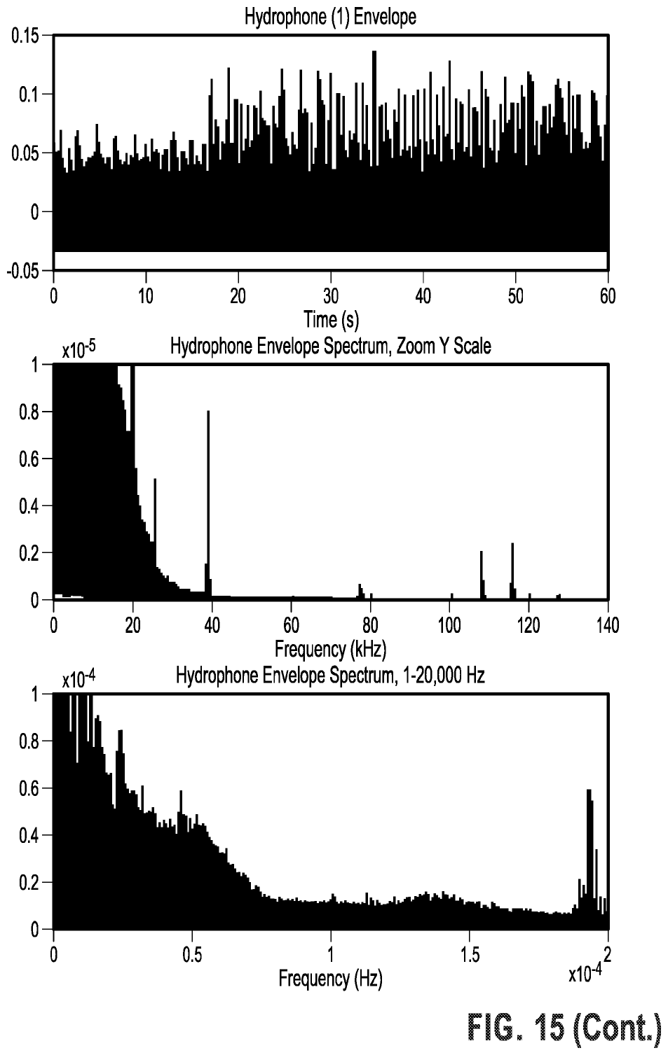

FIG. 15 depicts frequency envelope spectrum analysis 1500 of repeating signals acquired from a hydrophone. Where the analysis 1500 is performed on the hydrophone-derived signals, similar to the triaxial accelerometer (Z-axis), a range of frequencies is identified. In the very high frequency range, frequencies of interest predominantly in the 40, 80 and 100 to 120 KHz range are seen.

FIGS. 10-15 demonstrate that the Z-axis component of a triaxial accelerometer and the hydrophone are comparable in detecting repeating LVAD-derived very high frequencies of interest in the 30 to 60 KHz range, with additional bands of interest that can be detected by the hydrophone in the 70 to 120 KHz range (4 to 6 times above the limit of human hearing). This, as a result, yields two potential approaches in further development of the disclosed technology. The first is immediate utility of an internal triaxial accelerometer (Z-axis orientation) attached to the LVAD housing itself, which can also be incorporated in future LVAD designs. The second approach is a handheld surface detection device, where the hydrophone device works independent of plane or orientation, which can be used in clinical settings. The same techniques, processes, and methods described herein can be applied to both approaches to accurately detect pump thrombosis.

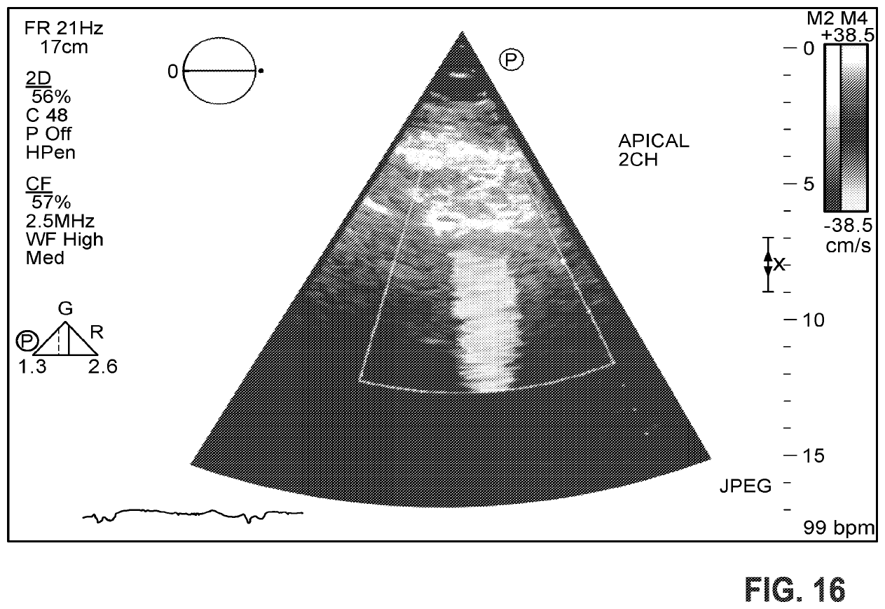

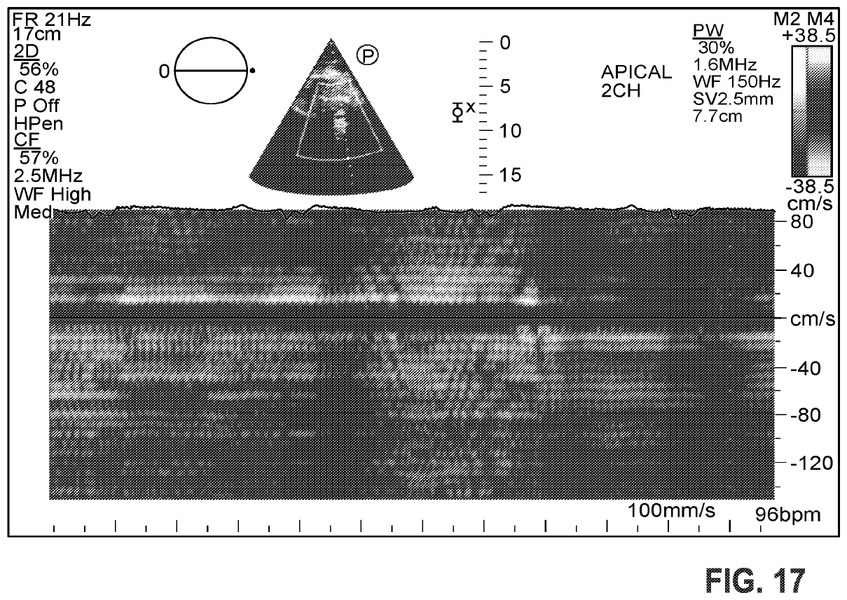

FIG. 16 is a 2D color doppler imaging 1600 in an apical 2 chamber view that demonstrates LVAD derived interference. FIG. 17 is a pulsed wave doppler imaging 1700 in an apical 2 chamber view that demonstrates waveform signals from the doppler interference identified in FIG. 16. Referring to both FIGS. 16 and 17, doppler imaging at echocardiography demonstrates evidence of electromagnetic interference from the LVAD in the apical views (refer to FIG. 16) which, on pulsed wave doppler imaging reveals waveforms consistent with a time-amplitude plot demonstrating the waveforms (refer to FIG. 17). It is of interest to subject these signals to time frequency analysis, as performed on the signals obtained utilizing the triaxial accelerometer and hydrophone described in reference to FIGS. 10-15. Performing time frequency analysis with the doppler imaging 1600 and 1700 can be advantageous to further define the frequencies of interest to target in development and refinement of devices employing the techniques described herein for early and accurate thrombosis detection.

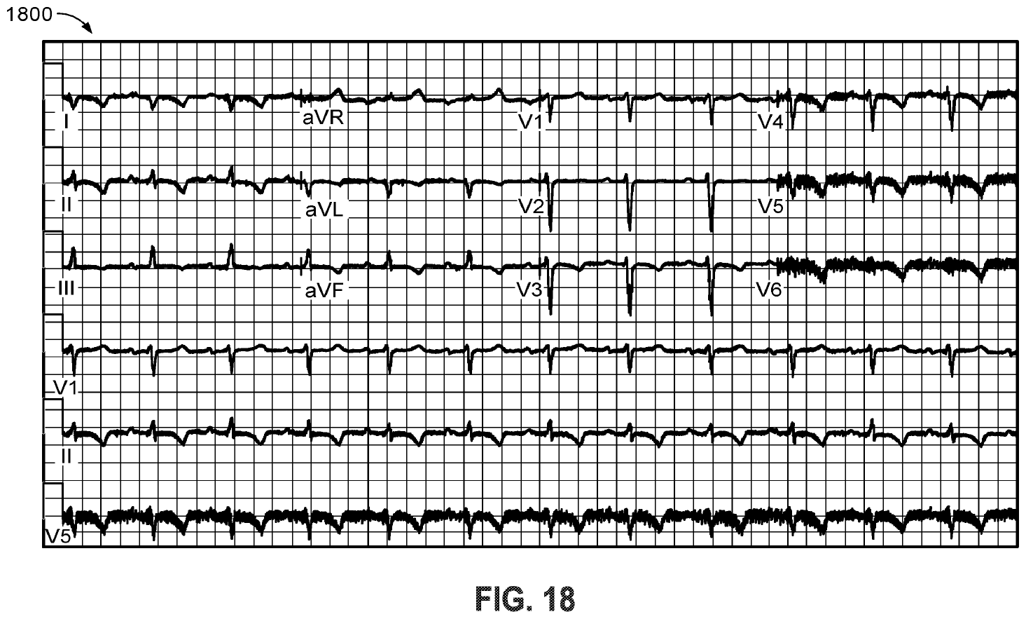

FIG. 18 depicts ECG 1800 demonstrating LVAD derived electrical noise. Similarly, LVAD derived interference is also seen on the ECG 1800, predominantly in centrifugal devices, such as the HeartWare HVAD and the HeartMate III LVAS. Time frequency analysis of the raw digital data from these ECGs 1800 can provide for identification and isolation of the LVAD derived electrical interference, and can be useful to make retrospective comparisons in patients who developed pump thrombosis of the electrical frequency distribution before and during onset of pump thrombosis. The detection of thrombosis may be enhanced by detection of alternation in specific frequencies identified with the onset of pump thrombosis during this retrospective analysis of the ECG 1800.

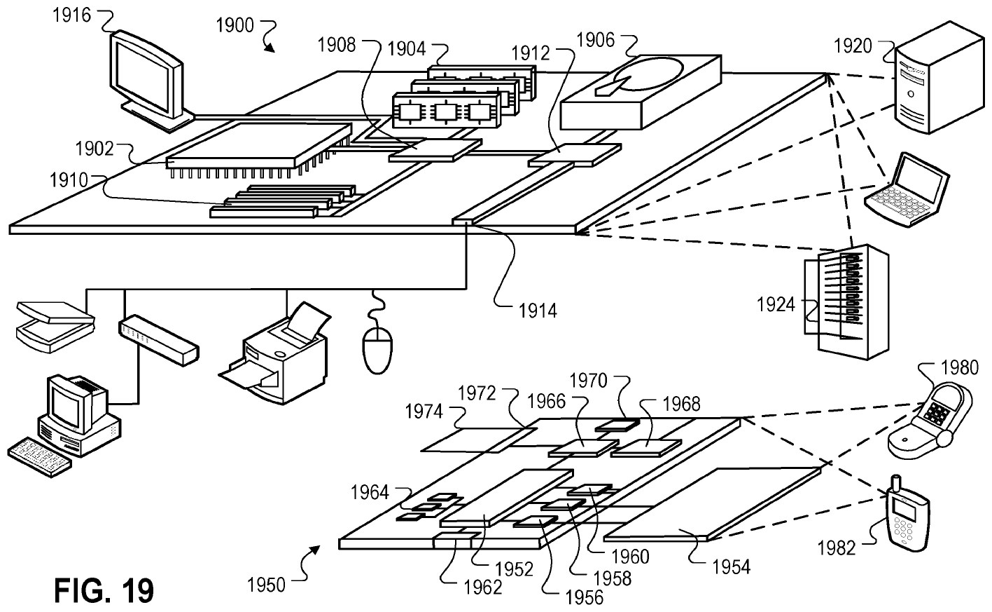

FIG. 19 shows an example of a computing device 1900 and an example of a mobile computing device that can be used to implement the techniques described here. The computing device 1900 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1900 includes a processor 1902, a memory 1904, a storage device 1906, a high-speed interface 1908 connecting to the memory 1904 and multiple high-speed expansion ports 1910, and a low-speed interface 1912 connecting to a low-speed expansion port 1914 and the storage device 1906. Each of the processor 1902, the memory 1904, the storage device 1906, the high-speed interface 1908, the high-speed expansion ports 1910, and the low-speed interface 1912, are interconnected using various busses, and can be mounted on a common motherboard or in other manners as appropriate. The processor 1902 can process instructions for execution within the computing device 1900, including instructions stored in the memory 1904 or on the storage device 1906 to display graphical information for a GUI on an external input/output device, such as a display 1916 coupled to the high-speed interface 1908. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1904 stores information within the computing device 1900. In some implementations, the memory 1904 is a volatile memory unit or units. In some implementations, the memory 1904 is a non-volatile memory unit or units. The memory 1904 can also be another form of computer-readable medium, such as a magnetic or optical disk.

The storage device 1906 is capable of providing mass storage for the computing device 1900. In some implementations, the storage device 1906 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1904, the storage device 1906, or memory on the processor 1902.

The high-speed interface 1908 manages bandwidth-intensive operations for the computing device 1900, while the low-speed interface 1912 manages lower bandwidth-intensive operations. Such allocation of functions is exemplary only. In some implementations, the high-speed interface 1908 is coupled to the memory 1904, the display 1916 (e.g., through a graphics processor or accelerator), and to the high-speed expansion ports 1910, which can accept various expansion cards (not shown). In the implementation, the low-speed interface 1912 is coupled to the storage device 1906 and the low-speed expansion port 1914. The low-speed expansion port 1914, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1900 can be implemented in a number of different forms, as shown in the figure. For example, it can be implemented as a standard server 1920, or multiple times in a group of such servers. In addition, it can be implemented in a personal computer such as a laptop computer 1922. It can also be implemented as part of a rack server system 1924. Alternatively, components from the computing device 1900 can be combined with other components in a mobile device (not shown), such as a mobile computing device 1950. Each of such devices can contain one or more of the computing device 1900 and the mobile computing device 1950, and an entire system can be made up of multiple computing devices communicating with each other.

The mobile computing device 1950 includes a processor 1952, a memory 1964, an input/output device such as a display 1954, a communication interface 1966, and a transceiver 1968, among other components. The mobile computing device 1950 can also be provided with a storage device, such as a micro-drive or other device, to provide additional storage. Each of the processor 1952, the memory 1964, the display 1954, the communication interface 1966, and the transceiver 1968, are interconnected using various buses, and several of the components can be mounted on a common motherboard or in other manners as appropriate.

The processor 1952 can execute instructions within the mobile computing device 1950, including instructions stored in the memory 1964. The processor 1952 can be implemented as a chipset of chips that include separate and multiple analog and digital processors. The processor 1952 can provide, for example, for coordination of the other components of the mobile computing device 1950, such as control of user interfaces, applications run by the mobile computing device 1950, and wireless communication by the mobile computing device 1950.

The processor 1952 can communicate with a user through a control interface 1958 and a display interface 1956 coupled to the display 1954. The display 1954 can be, for example, a TFT (Thin-Film-Transistor Liquid Crystal Display) display or an OLED (Organic Light Emitting Diode) display, or other appropriate display technology. The display interface 1956 can comprise appropriate circuitry for driving the display 1954 to present graphical and other information to a user. The control interface 1958 can receive commands from a user and convert them for submission to the processor 1952. In addition, an external interface 1962 can provide communication with the processor 1952, so as to enable near area communication of the mobile computing device 1950 with other devices. The external interface 1962 can provide, for example, for wired communication in some implementations, or for wireless communication in other implementations, and multiple interfaces can also be used.

The memory 1964 stores information within the mobile computing device 1950. The memory 1964 can be implemented as one or more of a computer-readable medium or media, a volatile memory unit or units, or a non-volatile memory unit or units. An expansion memory 1974 can also be provided and connected to the mobile computing device 1950 through an expansion interface 1972, which can include, for example, a SIMM (Single In Line Memory Module) card interface. The expansion memory 1974 can provide extra storage space for the mobile computing device 1950, or can also store applications or other information for the mobile computing device 1950. Specifically, the expansion memory 1974 can include instructions to carry out or supplement the processes described above, and can include secure information also. Thus, for example, the expansion memory 1974 can be provide as a security module for the mobile computing device 1950, and can be programmed with instructions that permit secure use of the mobile computing device 1950. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer-or machine-readable medium, such as the memory 1964, the expansion memory 1974, or memory on the processor 1952. In some implementations, the computer program product can be received in a propagated signal, for example, over the transceiver 1968 or the external interface 1962.

The mobile computing device 1950 can communicate wirelessly through the communication interface 1966, which can include digital signal processing circuitry where necessary. The communication interface 1966 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1968 using a radio-frequency. In addition, short-range communication can occur, such as using a Bluetooth, WiFi, or other such transceiver (not shown). In addition, a GPS (Global Positioning System) receiver module 1970 can provide additional navigation-and location-related wireless data to the mobile computing device 1950, which can be used as appropriate by applications running on the mobile computing device 1950.

The mobile computing device 1950 can also communicate audibly using an audio codec 1960, which can receive spoken information from a user and convert it to usable digital information. The audio codec 1960 can likewise generate audible sound for a user, such as through a speaker, e.g., in a handset of the mobile computing device 1950. Such sound can include sound from voice telephone calls, can include recorded sound (e.g., voice messages, music files, etc.) and can also include sound generated by applications operating on the mobile computing device 1950.

The mobile computing device 1950 can be implemented in a number of different forms, as shown in the figure. For example, it can be implemented as a cellular telephone 1980. It can also be implemented as part of a smart-phone 1982, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

1. A system for assessing a condition of a mechanical circulatory support device (MCSD) in a mammal, the system comprising:
a sensing subsystem configured to obtain from one or more sensors signals indicative of at least one of vibrations or acoustics of the MCSD in operation in the mammal; and
a computing subsystem configured to:
receive the signals indicative of at least one of the vibrations or the acoustics of the MCSD in operation in the mammal;
process the signals to identify at least one harmonic in the signals; and
evaluate the condition of the MCSD using the at least one harmonic identified in the signals indicative of at least one of the vibrations or the acoustics of the MCSD in operation in the mammal.

2. The system of claim 1, wherein the at least one harmonic is at least one of a fundamental frequency (FF), second harmonic (2H), third harmonic (3H), fourth harmonic (4H), sixth harmonic (6H), or a harmonic of higher-order than 6H.

3. The system of claim 1, wherein the one or more sensors include at least one of a microphone, a triaxial accelerometer, or a hydrophone.

4. The system of claim 1, wherein processing the signals comprises filtering extraneous noise from the signals.

5. The system of claim 1, wherein evaluating the condition of the MCSD comprises determining that an absolute amplitude of the at least one harmonic identified exceeds a threshold level.

6. The system of claim 5, wherein the at least one harmonic is at least one of a 2H or a 3H.

7. The system of claim 1, wherein evaluating the condition of the MCSD comprises determining that a relative amplitude of the signals exceeds a threshold level.

8. The system of claim 7, wherein the relative amplitude is a ratio of an amplitude of a 3H to an amplitude of a FF in the signals.

9. The system of claim 1, wherein the one or more sensors are positioned on the mammal's chest at a left lower sternal border.

10. The system of claim 1, wherein the one or more sensors are attached to a handheld surface detection device.

11. The system of claim 1, wherein the one or more sensors are attached to the MCSD.

12. The system of claim 1, wherein the one or more sensors include at least one of an electronic stethoscope, a hydrophone, an accelerometer, an ECG sensor, or an echocardiographic sensor.

13. The system of claim 1, wherein processing the signals to identify at least one harmonic in the signals comprises:
aurally identifying FF, 2H, and 3H as audible tones;
reproducing the audible tones into generated tones using a signal generator;
superimposing the generated tones into a sound recording;
fine tuning frequency of the generated tones in the sound recording by identifying beats;
identifying visual waveforms based on playing back the sound recording;
performing fast fourier transformation (FFT) on the sound recording;
labeling frequencies in the sound recording; and
labeling higher order harmonics that are inaudible but are identified based on performing the FFT.

14. The system of claim 1, wherein the computing subsystem is further configured to:
determine, based on evaluating the condition of the MCSD, that the MCSD has pump thrombosis; and
output, at a user device, an alert notification identifying the pump thrombosis of the MCSD.

15. The system of claim 1, wherein the MCSD is a left ventricular assist device (LVAD).

16. The system of claim 1, wherein the sensing subsystem is further configured to obtain, from the one or more sensors, signals indicative of electromagnetic signals of the MCSD in operation in the mammal.

17. The system of claim 1, wherein the computing subsystem is configured to evaluate the condition of the MCSD using the at least one harmonic identified based on:
retrieving, from a data store, one or more machine learning models; and
classifying, based on applying the one or more machine learning models, the at least one harmonic as corresponding to normal or abnormal operational conditions of the MCSD,
wherein the one or more machine learning models were trained using training datasets that include signals labeled with normal operational conditions and signals labeled with abnormal operational conditions.

18. The system of claim 1, wherein the abnormal operational conditions of the MCSD include at least one of a presence of pump thrombosis or a likelihood to develop pump thrombosis at the MCSD.

19. A method for assessing a condition of a mechanical circulatory support device (MCSD) in a mammal, the method comprising:
receiving, from one or more sensors, signals indicative of at least one of vibrations or acoustics of the MCSD in operation in the mammal;
processing the signals to identify at least one harmonic in the signals; and
evaluating the condition of the MCSD using the at least one harmonic identified in the signals indicative of at least one of the vibrations or the acoustics of the MCSD in operation in the mammal.

20. The method of claim 19, wherein evaluating the condition of the MCSD using the at least one harmonic identified comprises:
retrieving, from a data store, one or more machine learning models; and
classifying, based on applying the one or more machine learning models, the at least one harmonic as corresponding to normal or abnormal operational conditions of the MCSD,
wherein the one or more machine learning models were trained using training datasets that include signals labeled with normal operational conditions and signals labeled with abnormal operational conditions.
